(19) United States
(12) Patent Application Publication
(10) Pub. No.: US 2024/0242841 A1
(43) Pub. Date: Jul. 18, 2024

(54) SYSTEM AND METHOD FOR IDENTIFYING AND PREDICTING HYPOGLYCEMIA RISK

(71) Applicants: William B. HORTON, Charlottesville, VA (US); J. Randall MOORMAN, Keswick, VA (US); Matthew T. CLARK, Charlottesville, VA (US)

(72) Inventors: William B. HORTON, Charlottesville, VA (US); J. Randall MOORMAN, Keswick, VA (US); Matthew T. CLARK, Charlottesville, VA (US)

(21) Appl. No.: 18/551,055

(22) PCT Filed: Mar. 21, 2022
(86) PCT No.: PCT/US2022/021162

§ 371 Date: Sep. 18, 2023

Related U.S. Application Data
(60) Provisional application No. 63/163,480, filed on Mar. 19, 2021.

(51) Int. Cl. G16H 50/30; A61B 5/00; A61B 5/024; G16H 50/70

(52) U.S. Cl. CPC G16H 50/30; G16H 50/70; A61B 5/02405; A61B 5/7275

(57) ABSTRACT

Embodiments relate to systems and methods for predicting hypoglycemia risk via a predictive model. The method involves receiving a first set of patient data, applying data processing to identify features of the first set of patient data that are associated with hypoglycemia, applying multivariable modeling to the features to generate a multivariable model that outputs a risk score associated with future hypoglycemia where the multivariable model captures a pathophysiological signature of impending hypoglycemia. The method involves receiving a second set of patient data, applying data processing to identify features of the second set of patient data, and applying the multivariable model to generate a risk score for the second set of patient data. The method involves analyzing the risk score of the second set of patient data to determine an appropriate clinical decision support, and outputting a result for access by a device.

### CROSS-REFERENCE TO RELATED APPLICATIONS

This patent application is related to and claims the benefit of priority of U.S. Provisional Application No. 63/163,480, filed on Mar. 19, 2021, the entire contents of which is incorporated by reference.

### FIELD

Embodiments relate to a system and method of identifying and predicting hypoglycemia risk in real time by describing signatures, via multivariable analytics, of hypoglycemia from readily-available physiological and biochemical data.

### BACKGROUND INFORMATION

Methods and system exist that assess risk of hypoglycemia, but none incorporate hemodynamic and electrophysiological bedside monitoring data to provide a comprehensive and quantitative predictive model of the clinical pathophysiology of hypoglycemia.

### SUMMARY

An exemplary embodiment relates to a system for predicting hypoglycemia risk. The system has a processor with instructions to cause the processor to receive a first set of patient data. The instructions cause the processor to apply data processing to identify features of the first set of patient data that are associated with hypoglycemia. The instructions cause the processor to apply multivariable modeling to the features to generate a multivariable model that outputs a risk score associated with future hypoglycemia where the multivariable model captures a pathophysiological signature of impending hypoglycemia. The instructions cause the processor to receive a second set of patient data, apply data processing to identify features of the second set of patient data, and apply the multivariable model to generate a risk score for the second set of patient data. The instructions cause the processor to analyze the risk score of the second set of patient data to determine an appropriate clinical decision support. The instructions cause the processor to output a result for access by a device. At least one or more of the first set of patient data and/or the second set of patient data are representative of a physiological measurement from at least one or more of a cardiorespiratory monitoring (CRM) data source, an electronic medical record (EMR) vital sign data source, and/or a biochemical laboratory (LAB) data source.

An exemplary embodiment relates to a method for predicting hypoglycemia risk. The method involves receiving a first set of patient data. The method involves applying data processing to identify features of the first set of patient data that are associated with hypoglycemia. The method involves applying multivariable modeling to the features to generate a multivariable model that outputs a risk score associated with future hypoglycemia where the multivariable model captures a pathophysiological signature of impending hypoglycemia. The method involves receiving a second set of patient data, apply data processing to identify features of the second set of patient data, and apply the multivariable model to generate a risk score for the second set of patient data. The method involves analyzing the risk score of the second set of patient data to determine an appropriate clinical decision support. The method involves outputting a result for access by a device. At least one or more of the first set of patient data and/or the second set of patient data are representative of a physiological measurement from at least one or more of a cardiorespiratory monitoring (CRM) data source, an electronic medical record (EMR) vital sign data source, and/or a biochemical laboratory (LAB) data source.

An exemplary embodiment relates to a computer readable medium having instructions stored thereon that when executed by a processor causes the processor to predict hypoglycemia risk by receiving a first set of patient data. The processor predicts hypoglycemia risk b applying data processing to identify features of the first set of patient data that are associated with hypoglycemia. The processor predicts hypoglycemia risk by applying multivariable modeling to the features to generate a multivariable model that outputs a risk score associated with future hypoglycemia where the multivariable model captures a pathophysiological signature of impending hypoglycemia. The processor predicts hypoglycemia risk by receiving a second set of patient data, applying data processing to identify features of the second set of patient data, and applying the multivariable model to generate a risk score for the second set of patient data. The processor predicts hypoglycemia risk by analyzing the risk score of the second set of patient data to determine an appropriate clinical decision support. The processor predicts hypoglycemia risk by outputting a result for access by a device. At least one or more of the first set of patient data and/or the second set of patient data are representative of a physiological measurement from at least one or more of a cardiorespiratory monitoring (CRM) data source, an electronic medical record (EMR) vital sign data source, and/or a biochemical laboratory (LAB) data source.

### BRIEF DESCRIPTION OF THE DRAWINGS

Other features and advantages of the present disclosure will become more apparent upon reading the following detailed description in conjunction with the accompanying drawings, wherein like elements are designated by like numerals, and wherein:

FIG. 1 shows an exemplary system architecture for implementing an embodiment of the method;

FIGS. 2-3 show exemplary flow diagrams that may be used to build a predictive model;

FIG. 4-6 show exemplary data flow diagrams for an embodiment of the system;

FIG. 7 shows a data table of exemplary predictive models that can be generated and selected from for implementation;

FIG. 8 is a heat map depiction of the univariable risk of ICU hypoglycemia as a function of 61 measured physiologic and biochemical variables;

FIGS. 9A and 9B show cross-validated AUROC for the ICU hypoglycemia model;

FIGS. 10A and 10B show plotted calibration curve for the aggregate ICU hypoglycemia model; and

FIG. 11 is a block diagram illustrating an example of a machine upon which one or more aspects of embodiments of the present invention can be implemented.

### DETAILED DESCRIPTION

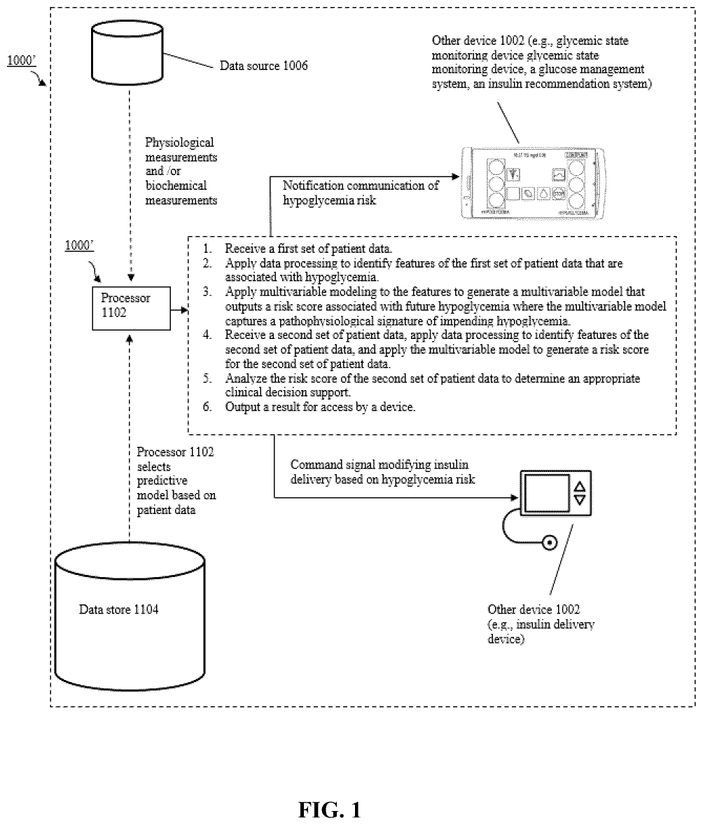

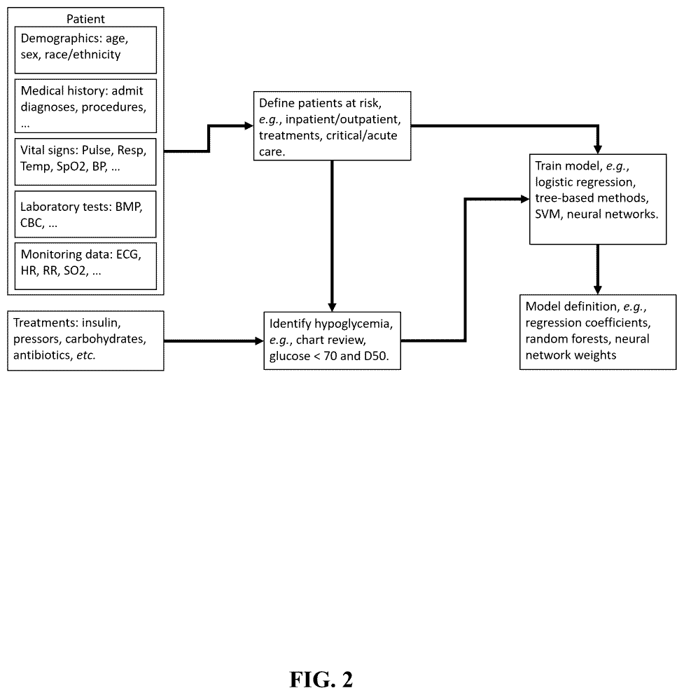

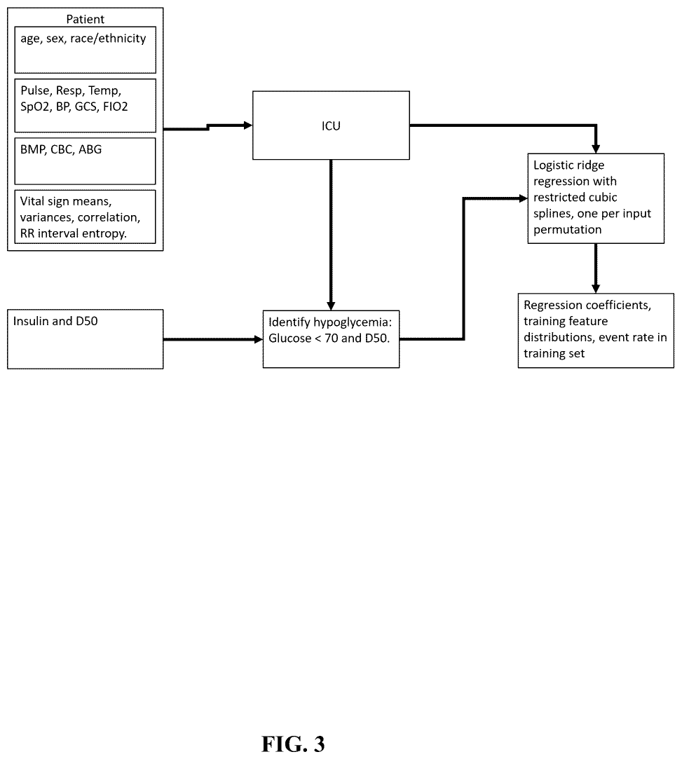

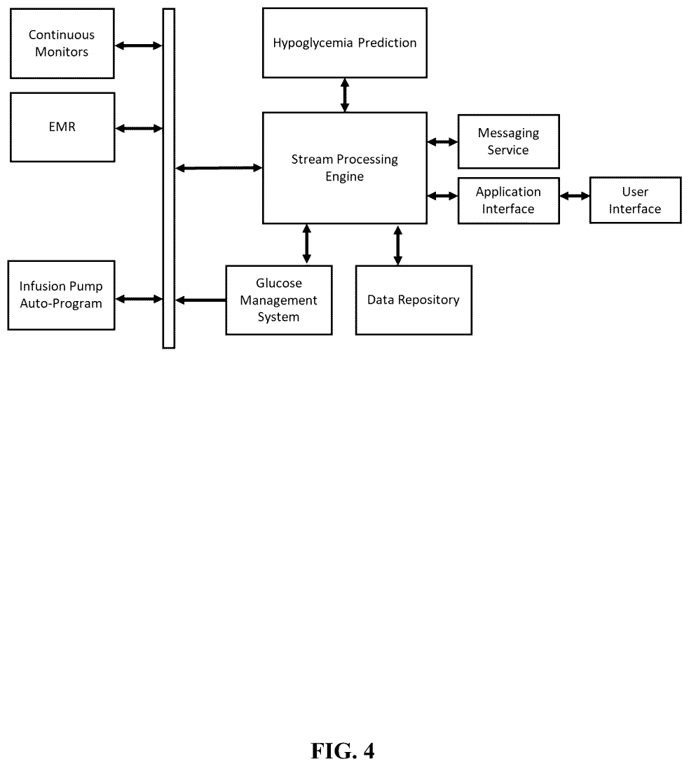

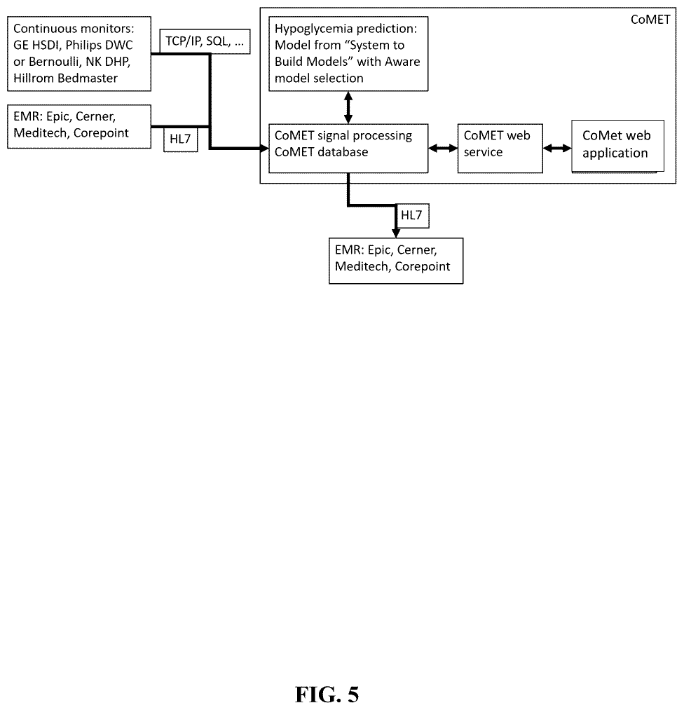

Referring to FIGS. 1-7, embodiments relate to a system 1000 for predicting hypoglycemia risk. The system 1000 includes a processor 1102 configured to build and implement a predictive model. The processor 1102 can be any of the processors 1102 disclosed herein. The processor 1102 can be part of or in communication with a machine 1100 (logic, one or more components, circuits (e.g., modules), or mechanisms). The processor 1102 can be hardware (e.g., processor, integrated circuit, central processing unit, microprocessor, core processor, computer device, etc.), firmware, software, etc. configured to perform operations by execution of instructions embodied in algorithms, data processing program logic, artificial intelligence programming, automated reasoning programming, etc. It should be noted that use of processors 1102 herein includes any one or combination of a Graphics Processing Unit (GPU), a Field Programmable Gate Array (FPGA), a Central Processing Unit (CPU), etc. The processor 1102 can include one or more processing modules. A processing module can be a software or firmware operating module configured to implement any of the method steps disclosed herein. The processing module can be embodied as software and stored in memory, the memory being operatively associated with the processor 1102. A processing module can be embodied as a web application, a desktop application, a console application, etc. Exemplary embodiments of the processor 1102 and the machine 1100 are discussed later.

The processor 1102 can include or be associated with a computer or machine readable medium 1122. As discussed in more detail later, the computer or machine readable medium 1122 can include memory. Any of the memory discussed herein can be computer readable memory configured to store data. The memory can include a volatile or non-volatile, transitory or non-transitory memory, and be embodied as an in-memory, an active memory, a cloud memory, etc. Embodiments of the memory can include a processor module and other circuitry to allow for the transfer of data to and from the memory, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc.

The computer or machine readable medium 1122 can be configured to store one or more instructions 1124 thereon. The instructions 1124 can be in the form of algorithms, program logic, etc. that cause the processor 1102 to build and implement a predictive model.

The processor 1102 can be in communication with other processors of other devices 1004 (e.g., a glycemic state monitoring device, a glucose management system, an insulin recommendation system, an insulin delivery device, etc.). Any of those other devices 1004 can include any of the exemplary processors 1102 disclosed herein. Any of the processors can have transceivers or other communication devices/circuitry to facilitate transmission and reception of wireless signals. Any of the processors can include an Application Programming Interface (API) as a software intermediary that allows two applications to talk to each other. Use of an API can allow software of the processor 1102 of the system 1000 to communicate with software of the processor of the other device(s) 1004.

The instructions 1124 to cause the processor 1102 to receive a first set of patient data. The first set of patient data can be historical, current, and/or real-time data. The first set of patient data is received by the processor 1102. This can be done continuously, periodically, or at some other predetermined schedule. The first set of patient data can be pulled by the processor 1102 from a data source 1006 and/or pushed from the data source 1006 to the processor 1102. The data source 1006 can be a device that measures the first set of patient data or a data store (e.g., database) that stores first set of patient data. The data source 1006 can be a cardiorespiratory monitoring (CRM) data source, an electronic medical record (EMR) vital sign data source, a biochemical laboratory (LAB) data source, etc. The first set of patient data includes data representative of a physiological measurement, medical history, nursing assessment, clinical intervention, and/or a biochemical measurement. Physiological measurements can include, for example, heart rate, blood glucose, blood pressure, respiration rate, body temperature, blood volume, sound pressure, photoplethysmography, electroencephalogram, electrocardiogram, blood oxygen saturation, skin conductance, etc. The physiological measurement can include waveform data related to heart rate, respiratory rate, electrocardiogramaspiratory effort, respiratory gas exchange, body impedance, and/or blood pressure. Nursing assessment can include patient alertness, cognition, pain scale, etc. Medical history can include medications used at baseline, supplemental oxygen requirements, diabetes history, blood cultures results, etc. Clinical interventions can include continuous insulin infusion dosages and rates, lines, drains, airways, antibiotics, etc. Biochemical measurements can include, for example, serum protein, serum micronutrient levels, serum lipids, and immunological parameters, albumin, prealbumin, hemoglobin, total iron-binding capacity, magnesium, vitamin levels, trace elements, cholesterol, triglycerides, fasting glucose, liver enzyme levels, etc.

The processor 1102 can store the first set of patient data in transient or persistent memory for later processing or process the patient data as it is being received. For instance, the processor 1102 can receive first set of patient data and aggregate the first set of patient data in storage. The aggregation can be based on the type of data, what the data represents, the time of receiving the data, the time the data was generated, etc., which can be embodied in metadata of the patient data. The processor 1102 can perform data processing to identify features of the first set of patient data that are associated with hypoglycemia. Features are variables or attributes of the first set of patient data, such as means, standard deviations, cross-correlations, entropy estimates, slopes, episodes of hypoglycemia, or encoded patient characteristics such as age, race, body temperature, pulse rate, etc. This can be done via feature selection or dimension reduction techniques such as fast backward elimination, principal component analysis, ridge regression, feature aggregation, etc. The analysis can involve identifying feature importance measures (e.g., correlation factors, covariance factors, goodness-of-fit, mean decrease in accuracy, Gini index, etc.) representative of each feature's association with hypoglycemia. The processor 1102 then stores these identified features in memory.

The processor 1102 can apply multivariable modeling techniques to the features to generate a multivariable model that outputs a risk score associated with future hypoglycemia. For instance, the multivariable model is configured to capture a pathophysiological signature of impending hypoglycemia—i.e., determine which features or combination of features statistically contribute to an increase in risk of hypoglycemia and map those features to a risk score that estimates the probability of impending hypoglycemia. There can be a risk score for each combination or permutation of features. The risk score can be any one of the individual risk scores or an aggregate (sum, average, weighted average, root-mean-square, etc.) of the plural risk scores. The risk score can indicate a risk of entering/exiting hypoglycemia or trending towards/away from hypoglycemia. The risk score can be from 0.0 to 1.0 for example. 0.0 can indicate little to no risk of hypoglycemia or little to no risk of entering hypoglycemia. 1.0 can indicate high risk of hypoglycemia or high risk of entering hypoglycemia. The risk score cane be relative to the average risk for example 1.0 means average risk, 2.0 means twice the average risk, etc. The multivariable model can be based on any one or combination of multivariable analyses. These can include logistic regression with or without cubic splines, random forest, xgboost, support vector machines, nearest neighbor, artificial neural networks, and/or long short-term memory (LSTM), multivariate analysis of variance (MANOVA), multivariate analysis of covariance (MANCOVA), principal components analysis (PCA), canonical correlation analysis, redundancy analysis (RDA), correspondence analysis (CA), canonical correspondence analysis (CCA), multidimensional scaling, discriminant analysis, linear discriminant analysis (LDA), clustering systems, recursive adaptive partitioning, vector autoregression, principal response curves analysis (PRC), etc. For example, the means, standard deviations, and cross correlations of heart rate, respiratory rate, and blood oxygen saturation can be fit with a logistic ridge regression model using cubic splines, the output of which is the probability of hypoglycemia in the next 8 hours.

The instructions 1124 to cause the processor 1102 to receive a second set of patient data. The second set of patient data is similar to the first set of patient data but is received at a later time. The first set of patient data is used to build the predictive model and the second set of patient data is used to asses risk of hypoglycemia for a patient(s) using the predictive model. The second set of patient data can be received continuously, periodically, or at some other predetermined schedule. The second set of patient data can be pulled by the processor 1102 from a data source 1006 and/or pushed from the data source 1006 to the processor 1102. The data source 1006 can be any of the data sources 1006 discussed herein. The second set of patient data can include any of the data types or measurements as those discussed above for the first set of patient data. The processor 1102 can store, process, aggregate, etc. the second set of patient data in any manner discussed above for the first set of patient data.

The processor 1102 can apply data processing to identify features of the second set of patient data. This can be done using any of the techniques discussed herein for the first set of patient data.

The processor 1102 can apply the multivariable model based on the first set of patient data to the features of the second set of patient data to generate a risk score for the second set of patient data.

The processor 1102 can analyze the risk score of the second set of patient data to determine an appropriate clinical decision support. The processor 1102 can output a result to a device 1002. For instance, a risk score of 0.0 can be used to generate a signal that no change is required. A risk score of 0.5 can be used by the processor 1102 to generate a signal requiring additional data to be obtained (e.g., a signal is generated requiring additional patient data, particular type of patient data, etc.), preventative or mitigating measures should be taken (e.g., a signal is generated to modify insulin rate, modify behavior, etc.), enhanced monitoring should be performed (e.g., a signal is generated to inform a user that the risk of hypoglycemia is heightened and additional monitoring should occur), etc. A risk score of 1.0 can be used by the processor 1102 to generate an alert signal, a command signal to an insulin device to modify insulin rate or dosage, etc. It is understood that other thresholds can be used and that the thresholds of 0.0, 0.5, and 1.0 are exemplary only. It is also understood that other scales can be used (e.g., the risk score can range from 0 to 10, 0 to 100, etc.) and that threshold could instead be applied to changes in scores (e.g., an increase of 50%). It is also understood that the processor 1102 can store the risk scores over time and generate a plot or trendline of the changes in risk score. This can be used for display to a user, for determining an appropriate clinical decision support, and/or for evaluating the effectiveness of interventions.

The system 1000 can include the processor 1102 alone (designated as 1000 in FIG. 1) or the processor 1102 in combination with one or more devices 1002 or other components (designated at 1000′ in FIG. 1). The processor 1102 in combination with a device 1002 can include the processor 1102 being part of the device 1002, the device 1002 being part of the processor 1102, the processor 1102 in communication with the device 1002, etc. “Being part of” can include being on a same substrate or integrated circuit. The device 1002 can be a glycemic state monitoring device, a glucose management system, an insulin recommendation system, etc. The device 1002 can be embodied as a computer device, a laptop, a cellphone, a smartphone, etc. The processor 1102 can be configured to generate a signal to inform the device 1002 about hypoglycemia risk based on the analysis of the risk score. For instance, the processor 1102 can generate a signal that includes a notification communication recommending, based on the analysis of the risk score, at least one or more of: risk of hypoglycemia, change in risk of hypoglycemia, check patient glucose level, modification of insulin dosage, modification of basal insulin, modification of basal insulin rate, modification of insulin infusion rate, and/or modification of patient nutritional administration. The type of signal, frequency (how often it is generated), the number of signals, etc. can depend on set thresholds and the risk of hypoglycemia (based on the risk score), change in risk of hypoglycemia (rate of change in risk score over a period of time), etc. The notification signal can be an email, short message service (SMS), a textual or graphical display, pager, etc.

In addition, or in the alternative, the system 1000 can be the processor 1102 in combination with a device 1002 that is an insulin delivery device. The processor 1102 can be configured to generate a signal to inform the insulin delivery device about hypoglycemia risk based on the analysis of the risk sore. For instance, the processor 1102 can generate a signal that includes a command signal requiring, based on the analysis of the risk score, at least one or more of: risk of hypoglycemia, change in risk of hypoglycemia, check patient glucose level, modification of insulin dosage, modification of basal insulin, modification of basal insulin rate, modification of insulin infusion rate, and/or modification of patient nutritional administration. The type of signal, frequency (how often it is generated), the number of signals, etc. can depend on set thresholds and the risk of hypoglycemia (based on the risk score), change in risk of hypoglycemia (rate of change in risk score over a period of time), etc.

As an exemplary example, risk score outputs of the processor 1102 can be useful in closed loop system by adjusting the rate of infusion of medications, such as insulin. In addition to using the blood or subcutaneous glucose level as inputs to an insulin delivery device 1002, the output of the risk model for hypoglycemia might also be used alone or in conjunction with the glucose levels. The extra information in the risk model (e.g., heart and respiratory rates, cardiorespiratory dynamics, and other factors such as medications and doses, and the times since the last feeding) add dimensions to the glucose levels alone. As can be appreciated by the present disclosure, the risk model is trained for detection of imminent events. This forecasting characteristic of the risk model adds information to the fleeting and, in the case of finger stick measurements, infrequent snapshot of the glucose levels.

It should be noted that the predictive model need not be limited to predicting the risk of future hypoglycemia. For instance, the patient data, data processing, and multivariable models can be configured to generate predictive models, the output being a risk score related to other physiological conditions (e.g., impending severe hypotension). Thus, the output can be used by the processor 1102 to adjust the rate of infusion of vasodilator medications in the treatment of hypertensive emergencies, vasoconstrictor medications in the treatment of hypotensive shock syndromes, etc. This can be complementary to use of blood pressure alone to adjust the medication rates, and in some instances be preferable, as the risk model integrates other factors such as heart and respiratory rates, cardiorespiratory dynamics, etc. to give a forecast of imminent deterioration.

The processor 1102 can be configured to generate the notification communication signal recommending modification of insulin infusion rate as a glucose clamp, wherein blood glucose is maintained within a range so as to bound blood glucose to an upper level and/or a lower level. Similarly, the processor 1102 can be configured to generate the command signal requiring modification of insulin infusion rate as a glucose clamp, wherein blood glucose is maintained within a range so as to bound blood glucose to an upper level and/or a lower level. Maintaining blood glucose can be done via insulin dosage rate, type of food intake, frequency of food intake, physical activity, etc. The notification communication signal or command signal can be configured to recommend or require any one or combination of these actions. With the clamp design, the processor 1102 can be in a closed-loop system with the other device 1002 so that the closed loop informs the other device 1002 of the physiological status of the patient. Information about the physiologic status of a patient is available from a closed-loop system that controls, for example, blood glucose. For a glucose clamp system, the insulin infusion rate can be automatically adjusted to keep the blood glucose within a specified range. This is achievable because the read-out of the amount of insulin required includes useful clinical information. The read-out can be used as an indicator of the level of illness because catecholamines and corticosteroids, the body's response to illness and stress, have anti-insulin properties. Thus, a rising level of insulin required to maintain normal glucose levels can inform of clinical deterioration.

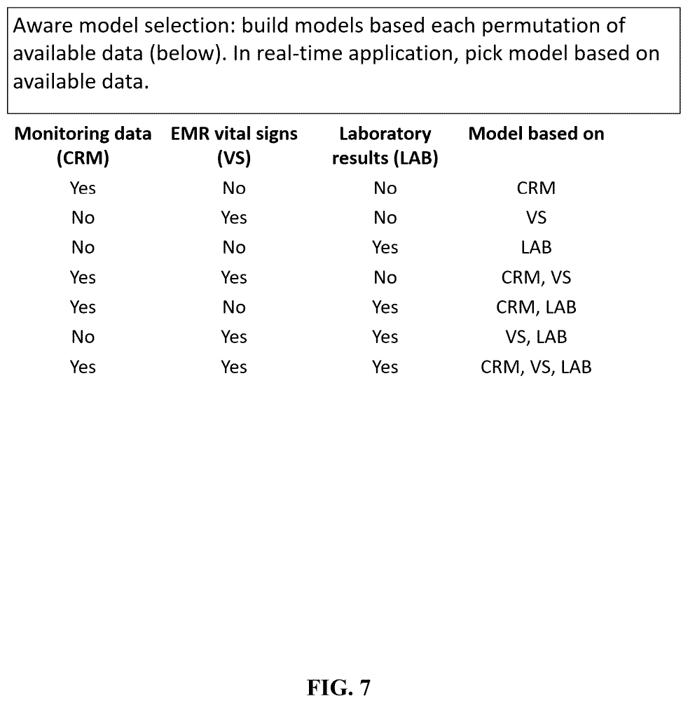

Referring specifically to FIG. 7, the system 1000 can include the processor 1102 in combination with a data store 1004. The data store 1004 can be configured to contain plural multivariable models. For instance, the system 1000 can be configured to generate plural multivariable models. The processor 1102 can be configured to implement any one or combination of the plural multivariable models. Each multivariable model can be generated based on the patient data available, the anticipated availability of patient data, the quality (how reliable the data is) of patient data, the frequency (how often it is generated or available) of patient data, dimensionality (how many attributes or variables the data has) of patient data, etc. For instance, a first multivariable model can be generated for a patient data set in which certain type of patient data is sparse but other type of patient data is abundant, a second predictive model can be generated for a patient set in which the reliability of certain data is low but is high for other type of patient data, etc. The type of patient data can include from which data source 1006 the data is received or attempted (or desired) to be received, which attributes are included in the data, the number of attributes the data has, etc. A multivariable model can be generated for anticipated patient data flows, thereby generating plural multivariable models. The plural multivariable models can be stored in a data store 1004. The processor 1102 can be in communication with a data store 1004 to as to access any one or combination of the plural multivariable models.

The processor 1102 is configured to select the multivariable model for implementation from the plural multivariable models based on at least one or more of: a type of first set patient data and/or a type of second set patient data. For instance, multivariable model-1 may be designed to better handle patient data that is abundant with CRM data but wanting regarding EMR vital sign data, multivariable model-2 may be designed to better handle patient data that is abundant with LAB data but wanting regarding CRM data, etc. The plural multivariable models can include at least one or more of: a CRM data model, an EMR vital sign data model, a LAB data model, a CRM/EMR vital sign data model, a CRM/LAB data model, an EMR vital sign/LAB data model, a CRM/EMR vial sign/LAB ddata model, etc. The processor 1102 can select the multivariable model best suited for the patient data being received. Again, the multivariable models generated and selected from can be based on which data source 1006 the data is received or attempted to be received, which attributes are included in the data, the number of attributes the data has, etc. Thus, the discussion herein regarding selection of predictive model based on data source 1006 is exemplary.

The processor 1102 can be configured to switch from a first multivariable model to a second multivariable model for implementation based on at least one or more of: a type of first set patient data and/or a type of second set patient data. Thus, if the patent data changes, the availability of the patient data changes, the reliability of the patient data changes, etc., the processor 1102 can detect the change (e.g., based on the metadata) and switch multivariable models.

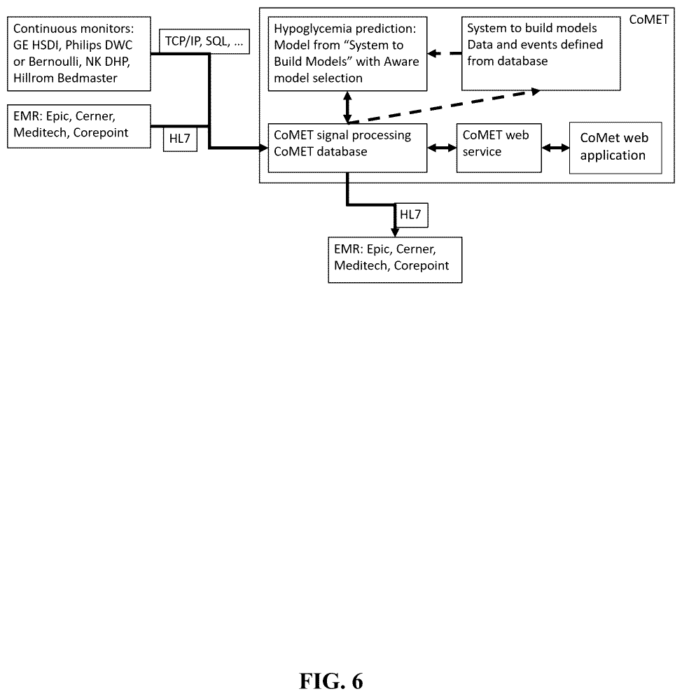

Referring specifically to FIG. 6, the processor 1102 can be configured to update the multivariable model based on patient data. As noted above, patient data is historical, current, and/or real-time data, and can be received continuously, periodically, or at some other predetermined schedule and can include information about hypoglycemic episodes and treatment thereof. The system 1000 can update any one or combination of multivariable models based on updated patient data. The updated multivariable model can replace the already existing multivariable model in the data store. Alternatively, if the updated multivariable model is sufficiently different or is better suited for a patient data scenario than any other existing multivariable model, the updated multivariable model can be added amongst the plural multivariable models.

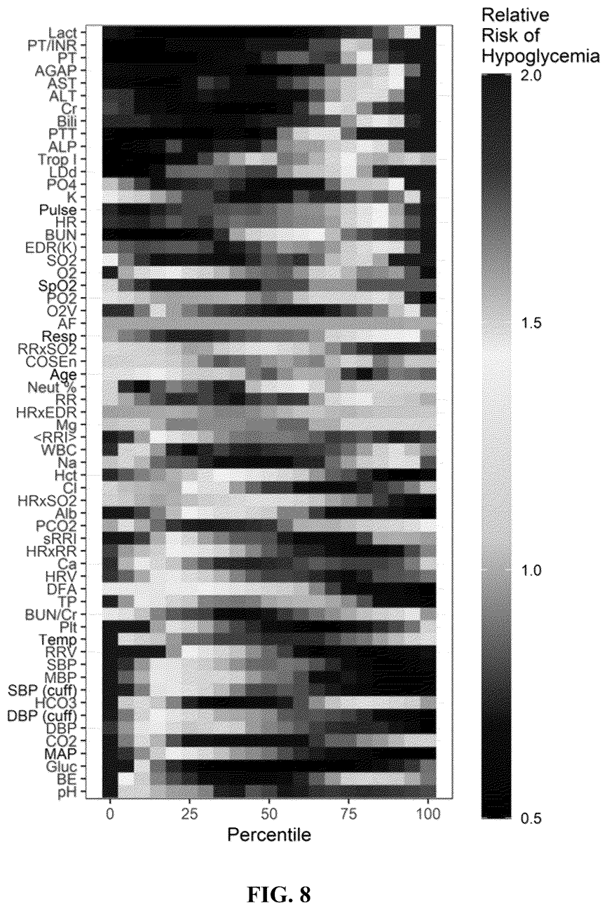

An exemplary method for predicting hypoglycemia risk can involve receiving a first set of patient data. The method can involve applying data processing to identify features of the first set of patient data that are associated with hypoglycemia, including cardiac entropy, blood pressure, respiratory rate, respiratory variability, and anion gap as in FIG. 8. The method can involve applying multivariable modeling to the features to generate a multivariable model that outputs a risk score associated with future hypoglycemia where the multivariable model captures a pathophysiological signature of impending hypoglycemia. For instance, the method can involve fitting a multivariable logistic regression model to these features to distinguish patient data within 8 hours of hypoglycemia from patient data far from hypoglycemia or in patient without hypoglycemia. The method can involve receiving a second set of patient data, applying data processing to identify features of the second set of patient data, and applying the multivariable model to generate a risk score for the second set of patient data. For instance, the method can involve applying the multivariable model prospectively to the features from new patients to generate a risk score associated with future hypoglycemia where the multivariable model captures a pathophysiological signature of hypoglycemia. The method can involve analyzing the risk score of the second set of patient data to determine an appropriate clinical decision support. The method can involve outputting a result for access by a device 1002. For instance, the method can involve sending the risk score to the electronic medical record to be included in patients' medical history, and send notifications to physician paging systems when the risk score indicates probability for impending hypoglycemia has acutely risen. At least one or more of the first set of patient data and/or the second set of patient data are representative of a physiological measurement from at least one or more of a cardiorespiratory monitoring (CRM) data source, an electronic medical record (EMR) vital sign data source, and/or a biochemical laboratory (LAB) data source.

The multivariable modeling can include at least one or more of: logistic regression, random forest, xgboost, support vector machines, nearest neighbor, artificial neural networks, and/or long short-term memory (LSTM).

Some embodiments can relate to a computer readable medium 1122 having instructions 1124 stored thereon that when executed by a processor 1002 causes the processor 1102 to predict hypoglycemia. The instructions 1124 cause the processor 1002 to receive a first set of patient data. The instructions 1124 cause the processor 1002 to apply data processing to identify features of the first set of patient data that are associated with hypoglycemia. The instructions 1124 cause the processor 1002 to apply multivariable modeling to the features to generate a multivariable model that outputs a risk score associated with future hypoglycemia where the multivariable model captures a pathophysiological signature of impending hypoglycemia. The instructions 1124 cause the processor 1002 to receive a second set of patient data, apply data processing to identify features of the second set of patient data, and apply the multivariable model to generate a risk score for the second set of patient data. The instructions 1124 cause the processor 1002 to analyze the risk score of the second set of patient data to determine an appropriate clinical decision support. The instructions 1124 cause the processor 1002 to output a result for access by a device 1002. At least one or more of the first set of patient data and/or the second set of patient data are representative of a physiological measurement from at least one or more of a cardiorespiratory monitoring (CRM) data source, an electronic medical record (EMR) vital sign data source, and/or a biochemical laboratory (LAB) data source.

The multivariable modeling can include at least one or more of: logistic regression, random forest, xgboost, support vector machines, nearest neighbor, artificial neural networks, and/or long short-term memory (LSTM).

As noted herein, the predictive model need not be limited to predicting the risk of future hypoglycemia. Patient data, data processing, and multivariable models discussed herein can be configured to generate predictive models, the output being used for predictive analytics monitoring related to coronavirus, sepsis, patient risk stratification, etc.

For instance, methods discussed in a) John P. Davis, MD, Dustin A. Wessells, and J. Randall Moorman, MD: Coronavirus Disease 2019 Calls for Predictive Analytics Monitoring-A New Kind of Illness Scoring System, Critical Care Explorations, 2020 and b) Oliver Monfredi, Jessica Keim-Malpass, and J Randall Moorman: Continuous cardiorespiratory monitoring is a dominant source of predictive signal in machine learning for risk stratification and clinical decision support, Physiological Measurement, 2021 (which are incorporated herein by reference in their entireties) can be used in conjunction with the predictive models disclosed.

Another example is heart rate characteristics monitoring for neonatal sepsis. In 2011, a multicenter group spearheaded at the University of Virginia demonstrated reduced mortality from real-time continuous cardiorespiratory monitoring in the neonatal ICU using what we now call artificial intelligence, Big Data, and machine learning. The large, randomized heart rate characteristics trial made real, for the first time that we know of, the promise that early detection of illness would allow earlier and more effective intervention and improved patient outcomes.

Currently, though, we hear as much of failures as we do of successes in the rapidly growing field of predictive analytics monitoring that has followed. This perspective aims to describe the principles of how we developed heart rate characteristics monitoring for neonatal sepsis and then applied them throughout adult ICU and hospital medicine. It primarily reflects the work since the 1990's of the University of Virginia group: the theme is that sudden and catastrophic deteriorations can be preceded by subclinical but measurable physiological changes apparent in the continuous cardiorespiratory monitoring and electronic health record.

The dream of anticipatory medicine is tantalizing but largely unrealized. We have pressing needs: patients are not only more numerous but also more ill, representing a population that would have died with usual care a decade or two ago. We also have expanding opportunities: clinical data are much more voluminous these days, presenting themselves in greater and greater variety and at higher and higher velocity. Unchanged, though, is the nature of day-to-day patient management. More than ever before, we spend our time reacting to in-the-moment catastrophic clinical deteriorations. While experienced clinicians agree that subtle premonitory changes can be apparent to the right eyes, automated detection of deterioration through sophisticated analysis of already available data is yet to transform the day-to-day practice of medicine. This notion, which we call predictive analytics monitoring, has been under fire recently: witness the poor performances of IBM Watson and the Epic Sepsis Model, and the finding that the “predictive” power of the electronic health record (EHR) lies mainly in what the physicians ordered, not results from the patients.

We fear that the haste to generate academic and commercial products has diverted focus toward the electronic health record (EHR)—a blurry image, at best, of the bedside—and away from the doctor, the nurse, the patient, and the continuous cardiorespiratory monitoring. Moreover, we believe that clinical points of view from the bedside have been subjugated to the perceived need for Big Data, so Big that the resolution of clinical definitions is lost. In order to realize fully the potential benefits of hospital-wide predictive analytics monitoring, we argue for a return to original principles, emphasizing clinical experience and reasoning, comprehensive and well-resolved data, sound mathematics, and the nuanced rigor of real-world practice.

### The Heart Rate Characteristics Monitoring Trial

In 2011 we published the results of one of the largest individually randomized clinical trials ever undertaken in premature infants. Previously, we found that premature infants who are early in the course of sepsis often have abnormal heart rate characteristics of reduced heart rate variability and transient decelerations. We developed or adapted mathematical time-series analytics that reflected the degree to which these abnormalities were present and mapped them to the probability of sepsis in the next 24 hours. We developed a logistics regression model adjusted for repeated measures and externally validated it at Wake Forest University. In the trial, we found that displaying a risk estimate based only on continuous cardiorespiratory monitoring streaming from the bedside monitors led to a more than 20% reduction in mortality. The only intervention was the display of the changing risk of sepsis: there were no alerts, alarms, thresholds, or mandated actions. The clinical benefits—lives saved, length of stay reduced, neurodevelopmental problems decreased—have been durable. The mechanism was as intended—infants with sepsis were saved.

### Principles Underlying the Development of Predictive Analytics Monitoring

The heart rate characteristics monitoring trial was one of the very earliest and most emphatic proofs of a general principle: predictive analytics monitoring saves lives by detecting subacute potentially catastrophic illness. The table (see Table I) recalls the properties we sought and the questions we asked when we developed predictive analytics monitoring for neonatal sepsis.

TABLE I
Properties and key questions for predictive analytics
monitoring research and development

| | |
|---|---|
| Clinical fit | If we detect the problem early, can we |
| | do something about it? |
| Face validity | Can we expect a subclinical prodrome |
| | detectable early on? |
| Signature of illness | Do we record the right signals? |
| Mathematical foundations | Do we analyze those signals in the right way? |
| Ground truth events | Is the model trained on all actual cases |
| | and no others? |
| Dynamicity | Does the risk estimate rise as the disease nears? |
| Clinical trial | Does it work in real life? |

### 1. Clinical Fit.

Sepsis is a common and potentially catastrophic illness, especially in premature infants where it greatly increases morbidity and mortality. The diagnosis is elusive because it presents with non-specific findings, but delaying antibiotics increases the death rate. The need for earlier detection has long been called for by authoritative groups such as the Neonatal Research Network of the NICHD.

Throughout the hospital, in fact, subacute, potentially catastrophic illnesses are common and have adverse outcomes. For example, we found that more than 10% of patients in a surgical and trauma ICU had at least one such event and that the impact on outcomes was outsized: several-fold increases in length of stay and even larger fold increases in death rates. Further, ward patients who deteriorated clinically and were transferred to ICUs had a 40-fold increase in mortality.

Perspective: Predictive analytics monitoring fits well clinically. It can meet a need for improved care in conditions where early detection might lead to earlier treatment which, in turn, might reasonably be expected to improve outcomes.

### 2. Face Validity.

Though it may present suddenly, sepsis in infants (and children and adults) is not a sudden illness, so we expect premonitory changes. When clinicians look back on septic patients for whom we made the diagnosis late, we can see subtle but consistent findings of rising heart rates, falling blood pressures, changing temperatures and white blood cell counts.

While sepsis is a flagship example, there are other subacute potentially catastrophic illnesses in which we can expect a subclinical prodrome. These include respiratory deterioration leading to emergency intubation, hemorrhage leading to large transfusion, hypoglycemia, and the multiple reasons that ward patients deteriorate and require ICU transfer. Their common characteristics are (1) a natural progression of physiological derangement that begins subtly, (2) a logical approach to diagnostic testing, and (3) therapy that is most effective early in the course of the illness. In our examples above, these include chest X-rays, bronchodilators, diuretics, antibiotics; or angiography, blood counts, surgery, transfusions; or fingersticks, feedings, glucose; or any of the many tests and treatments for the diverse and idiosyncratic modes of clinical deterioration. In each case, an early start to diagnosis and treatment seems likely to help some patients. These treatable conditions are better targets for early detection than, say, all-cause mortality within the following year, which has no clinical urgency, or ventricular fibrillation in the Coronary Care Unit, which has no prophylactic therapy. We note, though, that not all clinical deteriorations are of this kind. Some acute illnesses in the hospital are genuinely of sudden onset—vascular catastrophes like acute myocardial infarction, cerebrovascular accident, and pulmonary embolism, or arrhythmias such as ventricular fibrillation. For them, we expect no prodromes and no opportunities for forewarning. In fact, the absence of premonitory changes in the continuous cardiorespiratory monitoring delimits the differential diagnosis of sudden clinical deterioration.

Perspective: We expect subclinical prodromes for some subacute potentially catastrophic illnesses, so predictive analytics monitoring has face validity as a means for early detection.

### 3. Signatures of Illness

We built on the earlier observations of reduced heart rate variability in infants with respiratory distress. In premature infants with late-onset sepsis at the University of Virginia, we saw something new. Transient decelerations, many of them too small to generate a bradycardia alarm, punctuated the otherwise unvarying heart rate record. This is the same abnormality that distressed fetuses display, and it is perhaps not, after all, surprising that premature infants might report illness in the same way. This signature of acute neonatal illness applies not just to sepsis but also to necrotizing enterocolitis, respiratory distress, and bleeding. Note that this illness signature requires continuous cardiorespiratory monitoring to detect. It is not apparent by glancing at bedside monitors, nor is it captured in the EHR. Beaulieu-Jones and coworkers made the seminal observation that much of the predictive nature of the EHR lay in the orders placed by physicians on the day of admission. They pointed out that such clinician-initiated actions reflected the thinking of the physicians rather than findings from the patients, and thus that EHR-only-based statistical models might be lagging indicators rather than leading ones. Delays in recording vital signs 35 and in reporting lab results further increase the lag.

We endorse their view that telemetric real-time physiological monitoring is a source of non-clinician-initiated information that is more likely to reflect the patient's status. The notion resonates clinically—why would we not use the patient's physiologic data to make decisions about his or her physiologic status? We know well how the autonomic nervous system collects information from throughout the body and fine-tunes the heart and lungs in response, and a new body of knowledge points to the sophistication of regulation of the sinus node and the heartbeat by intrinsic mechanisms. While sensible to consider in any clinical setting, continuous cardiorespiratory monitoring data are rarely used in illness scoring systems despite adding information by themselves or by adding to the EHR. While we went on to find in the NICU that lab tests and clinical findings added independent information, we stand by the practice of always using continuous cardiorespiratory monitoring data wherever we find it in the hospital.

Perspective: Signatures of illness are better detected when we record the right signals, those that tell us more about the patient than the clinician. For this task, models that use continuous cardiorespiratory monitoring will always be better than those that don't.

### 4. Sound Mathematical Time Series Analysis and Statistical Modeling

The existing tools of heart rate variability analysis did not serve to detect records with abnormal heart rate characteristics because the decelerations inflate the standard deviation. We have used time-domain, frequency- and wavelet-domain, phase-domain, non-linear dynamical-domain, and other mathematical tools to characterize the dynamics of the heart and lungs from bedside continuous cardiorespiratory monitoring. Our final strategy comprised the standard deviation to detect long records with only reduced heart rate variability; sample asymmetry, new measures of the decelerations and accelerations; and sample entropy, which here serves to capture the phenotype of flat baselines with spikes.

These approaches have irrefutable mathematical foundations not subject to changing points of view. We note the promise of a comprehensive strategy of Fulcher and coworkers that they named highly comparative time-series analysis. Our recent work points to a reduced set of calculations on heart rate and oxygen saturation time series that captures many facets of cardiopulmonary physiology in premature infants.

These kinds of quantitative methods can reliably and reproducibly lead to the optimal development of features that relate physiologic dynamics to outcomes. Such mathematical approaches differ from point scores using thresholds picked by experts, such as the Apgar score, Score for Neonatal Acute Physiology (SNAP), or the Sequential Organ Failure Assessment (SOFA) and its neonatal version, APACHE, and others, all made problematic by the need for thresholds and dichotomization.

To optimize combinations of predictors, we have used mainly logistic regression in our work. We know of the proliferation of other machine learning and the newer recurrent neural network approaches of Deep Learning. (Indeed, we used a neural network in our first work on neonatal heart rate analysis in 1994.) While the newer approaches have revolutionized radiology with image analysis, we find no clear and consistent superiority of one method over another in this field of classifying the risk of patients from clinical data. We posit that newer machine learning and deep learning approaches should complement rather than replace traditional statistical pattern recognition methods.

Perspective: Once armed with the right signals, we should exercise the right analytic methods to quantify what they are telling us, ones that assay the physiological dynamics of the patient.

### 5. Ground Truth Cases in the Training Sets

Chart review by clinicians is the gold standard for identifying cases on which to train statistical models. This observation stands to reason clinically, and multiple studies have quantified the shortcomings of automated detection strategies for infection. There are two—failure to include cases in the training set, and dilution by non-cases. The impact depends on how the sensitivity and positive predictive accuracy compare to the incidence rate of the event. Say a good computer strategy for identifying events from the medical records has 70% sensitivity and 70% positive predictive accuracy, but the event rate is only 1%. In that case, a study of 10,000 patients identifies 70 of the 100 events, reducing the richness of the training set, and includes 30 patients without the event, diluting the training set by nearly half with irrelevant cases. In addition to concerns about the robustness and precision of models trained on impure data sets, the new focus on explainability is endangered. Confusion will follow when trying to understand the attributes of patients who did not have the targeted condition and failing to identify the attributes of those who did.

Perspective: It seems inarguable to us that models trained on all the actual cases and no others will always be better than those that aren't.

6. Dynamics of the Model that Match the Course of the Illness.

While statistical testing of the performance of the heart rate characteristics index was essential, there should be more to it than threshold-based evaluations like sensitivity and specificity or even areas under curves that evaluate multiple thresholds. (When a patient says s/he feels unwell, do you ask about their predictive performance?) We find that inspecting the time course of the model prediction as a function of the time until the event tells us much about what clinicians would see at the bedside. The phenotypes of the trajectories can say a great deal about the patient's prognosis. For example, we identified trajectories of heart rate characteristics monitoring that differentiated septic patients into higher and lower risk categories, a result presaged as long ago as 2003. Indeed, it is often the trend over time more so than the magnitude of the risk that leads clinicians to act. While highly problematic statistically, alerts based on threshold-crossings are not without value.

Indeed, the field of predictive analytics monitoring was recently advanced by Escobar and coworkers at Kaiser-Permanente who broadly adopted a very successful systems approach of alerts and informed intermediaries to reduce mortality at 19 hospitals. But the problems of alert fatigue are well-known, and few risk estimates have true thresholds, where the risk steps up but is constant on either side of the breakpoint.

Perspective: Illnesses are dynamic, and the risk estimate should rise as the signature becomes more clear.

### 7. A Large Randomized Clinical Trial

While RCTs have been criticized for expense, failure of scope, and limited applicability to clinical practice, the design remains inarguably persuasive. While new designs are welcome, the individually randomized trial remains a gold standard required to alter practice for many clinicians. The trial results overcome questions about metrics such as sensitivity and specificity, and are antidotes to anecdotal reports.

For example, there were important reassurances in the heart rate characteristics trial about the possibility of increased sepsis work-ups. To be sure, since the event is rare, most positives are false, and a review of a small subset of heart rate characteristics scores from one center had a negative conclusion. We found, though, no significant increase in blood cultures or antibiotics. We can surmise that low risk scores must have averted about as many sepsis work-ups and rule-outs as high scores initiated, an opinion voiced by practitioners in the study. This property of predictive analytics monitoring to reassure clinicians about the low-risk patients as well as to alert them to the high-risk ones is an additional utility not contemplated initially but emphatically present in the statistical analysis.

Perspective: Randomized clinical trials of predictive analytics monitoring in the real world remain of premium value. Unless repeated, there can be no gainsaying the result.

### Current and Future Directions

A new area of work is implementing and integrating predictive analytics monitoring into the complex arena of clinical care. We note a bare-bones education in the neonatal ICU and an organic spread of its use mainly driven by word of mouth. Our current implementation efforts in adult ICUs and wards of two hospitals and an eICU employ a systematic and principled approach, and we note the applicability of the monitoring to a learning health systems approach. Another new area is algorithmic equity. We propose that continuous cardiorespiratory monitoring may be less biased than the EHR as a data source, though work remains to test the idea. The interpretability of models is another desirable feature. We found that physicians and other clinicians wish to know the origins of rising risk as estimated by computers. Finally, we anticipate studies of the utility of Deep Learning on the continuous cardiorespiratory monitoring time-series data, where new patterns undetected by domain experts might yet be found.

### Limitations of Predictive Analytics Monitoring

Statistical models do not make diagnoses or tell us what to do next—all they can do is relate data to probability. It stands to reason that more data in more dimensions will improve the risk estimate, especially if the sampling is continuous, like bedside cardiorespiratory monitoring. Barriers to universal monitoring of hospital patients include the cost and the cumbersome nature of the devices. Several trends may change this picture. The pandemic has threatened the number of bedside clinicians who now serve to monitor patients closely, and technological advances have resulted in remarkably capable wearable devices that serve as cardiorespiratory monitors. Some day, perhaps one may need only to put an app on a watch to benefit from predictive analytics and other forms of continuous monitoring in the hospital.

Here is a more critical limitation: the data collected may not accurately paint the clinical picture of the patient. Like pointillism, a larger number of data points, and more strategically placed ones, better capture the identity of the illness. For a given patient, different clinicians might order different tests if their differential diagnoses differed. Each of the resulting data sets partially captures a competing view of the patient, further complicating the problem of making a statistical model for the classification of future patients. In the worst-case scenario, if a patient has sepsis but the chart has no recorded vital signs, labs, or other relevant data, then no scoring system can make an assessment. Beam and coworkers recently addressed the scenario when the predictive model has nothing to say on the matter.

A potential limitation of predictive analytics monitoring is that a blank EHR record cannot assess the patient in the present, let alone for the future.

We began our predictive analytics monitoring work more than 20 years ago by focusing on neonatal sepsis, a common and deadly illness with a sub-clinical prodrome and a signature of illness in continuous cardiorespiratory monitoring. We used mathematics to analyze non-clinician-initiated data in ground truth cases. The population- and illness-specific predictor changed dynamically with the risk of imminent illness, and its use improved outcomes in a large randomized trial. We believe that heart rate characteristics monitoring for neonatal sepsis is the earliest success of predictive analytics monitoring for subacute potentially catastrophic illness.

We offer this perspective as the template for our ongoing predictive analytics monitoring research, development, and implementation throughout the hospital. The guiding principles call for continuous cardiorespiratory monitoring, predictive models tailored for conditions and populations rather than just one model for the whole hospital, models trained on clinician-identified cases, sound mathematical foundations, display of changing risks rather than sounding alarms and alerts, and detailed schemes for implementation and integration that meld the predictive monitoring into the complex world of the hospital bedside.

The following discussion is an exemplary implementation of the disclosed method and results demonstrating the effectiveness of the disclosed method.

We tested the hypothesis that routine monitoring data could describe a detailed and distinct pathophysiologic phenotype of impending hypoglycemia in adult intensive care unit patients.

### Design:

Retrospective analysis leading to model development and validation.

### Setting/Patients:

All intensive care unit admissions wherein patients received insulin therapy during a four-year period at the University of Virginia Medical Center. Each intensive care unit was equipped with continuous physiological monitoring systems whose signals were archived in an electronic data warehouse along with the entire medical record.

### Interventions:

The primary outcome was hypoglycemia, defined as any episode of blood glucose <70 mg/dL where dextrose (i.e., D50) was also administered within one hour. We used 61 physiological markers (including vital signs, laboratory values, demographics, and continuous cardiorespiratory monitoring variables) to inform the model.

### Measurements and Main Results:

Our dataset consisted of 11,847 intensive care unit patient admissions, 721 (6.1%) of which had one or more hypoglycemic episodes. Multivariable logistic regression analysis revealed a pathophysiologic signature of 41 independent variables that best characterized intensive care unit hypoglycemia. The final model had a cross-validated AUROC of 0.83 (95% CI: 0.78-0.87) for prediction of impending intensive care unit hypoglycemia. We externally validated the model in the MIMIC-III critical care dataset, where it also demonstrated good performance with an AUROC of 0.79 (95% CI: 0.77-0.81).

### Conclusions:

We used data from a large number of critically-ill inpatients to develop and externally validate a predictive model of impending intensive care unit hypoglycemia.

### INTRODUCTION

Hypoglycemia, defined as a blood glucose level <70 mg/dL (3.9 mmol/L), is the most common side-effect of treatment for all types of diabetes and hyperglycemia in the hospital setting (1, 2). Inpatient hypoglycemia is associated with a number of adverse events, including patient distress, cardiac arrhythmias, cardiac ischemia, seizures, brain damage, increased length-of-stay, and increased short- and long-term mortality (1, 3-7). Beyond poor clinical outcomes, inpatient hypoglycemia also carries financial implications.

A recent study of 43,659 admissions within the Florida Hospital System found that even one episode of hypoglycemia resulted in a total cost of care that was $10,405 greater than in patients whose blood glucose remained within the normal range (8). With these factors in mind, The Centers for Medicare and Medicaid Services (CMS) has identified inpatient hypoglycemia as a high-priority measurement area and is currently adapting a hypoglycemia prevention measure (NQF #2363: Glycemic Control-Hypoglycemia) for possible future CMS use. In practice, this measure would incentivize hospitals to implement clinical workflows that facilitate evidence-based glycemic management strategies to reduce the likelihood of hypoglycemia events.

The prevalence of inpatient hypoglycemia is nearly threefold higher in the intensive care unit (ICU) than non-ICU settings (9, 10), and multiple studies confirm that ICU hypoglycemia is linked to increased morbidity and mortality (6, 11-13). Given the strong association between ICU hypoglycemia and poor outcomes, a proactive approach using targeted predictive analytics is needed (14). One such approach is to retrospectively analyze historical clinical data and develop a prediction tool that determines the individualized risk of ICU hypoglycemia. The possibility of developing such a prediction tool lies in the growing availability of rich clinical datasets stored in a hospital's electronic health records (EHR) system (15). With the well-established biochemical, hemodynamic, and electrophysiological changes that occur during hypoglycemia (16), EHRs provide an invaluable resource for prediction tool development. Despite recent advancements in EHRs and machine learning, few studies have focused on model development solely for ICU hypoglycemia (17).

In this study, we used machine learning to test the hypothesis that routine monitoring data could describe a detailed and distinct pathophysiologic phenotype of impending hypoglycemia in adult intensive care unit (ICU) patients. Our work led to the development and validation of a predictive model for ICU hypoglycemia that may provide a basis for future real-time predictive modeling that will improve recognition of impending hypoglycemia and direct earlier administration of preventive therapy in ICU patients.

### Materials and Methods

### Study Design

We retrospectively analyzed all ICU admissions from October 2013 to August 2017 at the University of Virginia (UVa) Medical Center wherein patients were ≥18 years old and received insulin therapy. This included medical (28 beds), surgical-trauma (15 beds), thoracic-cardiovascular postoperative (19 beds), coronary care (10 beds), and neuroscience (12 beds) ICUs. Each ICU was equipped with continuous physiological monitoring systems whose signals were archived in an electronic data warehouse along with the entire medical record. Monitoring data archival was interrupted in the coronary care and thoracic-cardiovascular postoperative ICUs in July 2015 due to changes in biomedical engineering infrastructure. We used the Transparent Reporting of a multivariable prediction model for Individual Prognosis or Diagnosis (TRIPOD) guidelines (18) and followed the recommendations set forth by Leisman et al. (19) to analyze and report this study (20). The UVa Institutional Review Board approved this study with a waiver of informed consent.

### Outcome Definition

The primary outcome was hypoglycemia, defined as any episode of blood glucose <70 mg/dL where dextrose (i.e., D50) was also administered within one hour. This specific definition was chosen because our EHR hypoglycemia order set includes administration of D50 whenever a blood glucose <70 mg/dL is recorded. Secondary outcomes included mortality and length-of-stay. We focused on physiological data starting 12 hours before the hypoglycemic episode. As controls, we included data from >12 hours before the hypoglycemic episode, and from insulin-treated ICU patients who did not experience hypoglycemia during admission. We censored data that followed each hypoglycemic episode.

### Model Development and Validation

We performed modeling in R 4.0.2 (R Core Team 2020; Vienna, Austria) using the “rms” package (21). For the univariable analysis, we plotted predictiveness curves to show the individual association of 61 vital signs, laboratory values, demographics, and continuous cardiorespiratory monitoring variables with episodes of hypoglycemia compared to no hypoglycemia. We randomly sampled one measurement within 24 hours before to 15 hours after each episode. We calculated the relative risk of hypoglycemia at each decile of the sampled variable and then interpolated the risk to 20 points evenly spaced in the range of the variable. We repeated this process of sampling, calculating relative risk, and interpolating 30 times. Finally, we averaged the 30 risk estimate curves to obtain a predictiveness curve at the 20 evenly-spaced points and displayed results as a heat map.

For multivariable modeling (both for the aggregate ICU model and the individual ICU models), we assessed 41 physiological variables that were at least 70% available and clinically relevant to hypoglycemia. We used multivariable logistic regression adjusted for repeated measures to relate physiological data to the hypoglycemia outcome on the entire cohort (21). We systematically built the model by: (1) removing, blinded to the outcome, the most predictable features correlated more than R2 of 0.9 with other features; (2) imputing missing values with median values for the study population; (3) building a model with all remaining features and restricted cubic splines (3 knots) on each feature with enough unique values (21, 22), adjusting for repeated measures using the Huber-White method (21); and (4) using ridge regression (23) to penalize model coefficients, shrinking the effective degrees of freedom to maximize the corrected AIC (24, 25). We then performed internal validation using 10-fold cross-validation (TRIPOD type 1b model study) (20, 26). We randomly split the patient-admissions into 10 groups, excluded a single group's data as a test set, trained a model on the remaining data using the same features and penalty found above, used that model to estimate the risk of hypoglycemia for the test set, and then calculated the area under the receiver operating characteristic curve (AUROC) in the test set. We repeated this procedure until each of the 10 groups had served as a test set, and used the 10 resulting AUROC measurements to estimate the mean and 95% confidence intervals. Although this method calculated out-of-sample predictions with the same features, we made the predictions with slightly different models, one for each test set.

External validation of the derived model was performed in the Medical Information Mart for Intensive Care (MIMIC-III) database (version 1.4), which is a freely available critical care dataset for researchers consisting of >40,000 ICU admissions (medical, surgical, coronary care, and neonatal) at Beth Israel Deaconess Medical Center (Boston, MA) from June 2001 to October 2012 (27). The MIMIC-III database was approved by the Institutional Review Boards of the Massachusetts Institute of Technology (Cambridge, MA) and Beth Israel Deaconess Medical Center (Boston, MA). The MIMIC-III cohort used in our analysis consisted of all adult ICU admissions.

### Results

### Baseline Characteristics and Outcomes

We analyzed data obtained from 11,847 UVa ICU patient admissions, 721 (6.1%) of whom had one or more hypoglycemic episodes. Table II demonstrates baseline demographics, admission characteristics, mortality, and length-of-stay for our study population. Notably, mortality was about threefold higher (28.3% vs. 9.8%; p-value <0.001) and length-of-stay doubled (15 days vs 7 days; p-value <0.001) in those who experienced hypoglycemia (Table II).

TABLE II
Demographic and clinical characteristics of critically-ill adult
patients admitted to the ICU from October 2013 to August 2017.

| | Hypo- | No | |
|---|---|---|---|
| | glycemia | Hypoglycemia | P- |
| Variable | (n = 721) | (n = 11,126) | value |
| Age, median | 62.7 | 63.6 | 0.019 |
| years (IQR) | (51.1-72.1) | (53.2-73.1) | |
| Sex, n (%) | | | |
| Male | 405 (56.2) | 6008 (54.0) | |
| Female | 316 (43.8) | 5118 (46.0) | 0.255 |
| Race, n (%) | | | |
| White | 536 (74.3) | 9098 (81.8) | <0.001 |
| Black | 169 (23.4) | 1744 (15.7) | <0.001 |
| Other | 10 (1.4) | 192 (1.7) | 0.496 |
| Asian | 5 (0.7) | 71 (0.6) | 0.857 |
| Unspecified | 0 (0.0) | 17 (0.2) | 0.294 |
| Native American | 1 (0.1) | 4 (0.0) | 0.193 |
| Weight, median | 80.3 | 83.5 | <0.001 |
| kilograms (IQR) | (64.9-97.1) | (71.2-99.8) | |
| Intensive Care | | | |
| Unit, n (%) | | | |
| CCU | 47 (6.5) | 752 (6.8) | 0.803 |
| MICU | 388 (53.8) | 3252 (29.2) | <0.001 |
| NNICU | 74 (10.3) | 3983 (35.8) | <0.001 |
| STICU | 141 (19.6) | 1771 (15.9) | 0.010 |
| TCVPO | 71 (9.8) | 1368 (12.3) | 0.051 |
| Length-of-Stay, | 15 (8-28) | 7 (4-12) | <0.001 |
| median days (IQR) | | | |
| Mortality, n (%) | 204 (28.3) | 1090 (9.8) | <0.001 |
| APACHE Score, | 14 (8-21) | 8 (4-14) | <0.001 |
| median (IQR) | | | |

(CCU = Coronary Intensive Care Unit;
MICU = medical intensive care unit;
NNICU = Neuroscience Intensive Care Unit;
STICU = Surgical-Trauma Intensive Care Unit;
TCVPO = Thoracic Cardiovascular Post-Operative Unit;
APACHE = Acute Physiology and Chronic Health Evaluation;
IQR = interquartile range).

Hypoglycemia was associated with increased mortality even after accounting for age, comorbidities, illness severity, and clinical presentation (p<0.0001). For external validation in MIMIC-III, we analyzed data obtained from 9,878 ICU patient admissions. Four hundred ninety-three (5.0%) of these ICU patient admissions experienced one or more hypoglycemic episodes. As in the UVA dataset, those who experienced hypoglycemia had higher mortality (27.6% vs. 10.7%; p-value <0.001) and longer length-of-stay (13.5 days vs 7.1 days; p-value <0.001) when compared to those who did not experience hypoglycemia.

### Pathophysiologic Signature of Impending Hypoglycemia

Univariable analysis of 61 physiological and biochemical variables identified trends in each that were associated with hypoglycemia (FIG. 8). FIG. 8 is a heat map depiction of the univariable risk of ICU hypoglycemia as a function of 61 measured physiologic and biochemical variables. Note, the heat map was generated in color but is presented here in grayscale. One skilled in the art would understand how to interpret the heat plot and equally understand what the results represent by using the grayscale. Each tile plots the value of the variable on the x-axis against the relative risk of ICU hypoglycemia on the y-axis. Variables on the y-axis represent model outputs, indicating laboratory values, hemodynamic monitoring variables, and electrophysiological variables. The relative risk bar ranges from 0.50 to 2.0, representing higher relative risk of hypoglycemia and lower relative risk. (Lact=lactate; PT/INR=prothrombrin time/international normalized ratio; AGAP=anion gap; AST=aspartate aminotransferase; ALT=alanine aminotransferase; Cr=creatinine; Bili=bilirubin; PTT=partial thromboplastin time; ALP=alkaline phosphatase; Trop I=troponin I; LDd=local dynamics density of heart rate; PO4=phosphorous; K=potassium; Pulse=heart rate measured by pulse oximetry (beats/min); HR=heart rate measured by cardiac telemetry (beats/min); BUN=blood urea nitrogen; EDR=electrocardiogram-derived respiratory rate (breaths/min); SO2=oxygen saturation measured by pulse oximetry (%); O2=oxygen saturation from arterial blood gas; SpO2=clinician-documented oxygen saturation (%), PO2=partial pressure of oxygen from arterial blood gas; O2V=the standard deviation of oxygen saturation by pulse oximetry; AF=probability of atrial fibrillation; Resp=respiratory rate measured by pulse oximetry; RRxSO2=cross-correlation coefficient of respiratory rate measured by chest impedance and oxygen saturation measured by pulse oximetry; COSEn=coefficient of sample entropy of R-R interval; Neut %=neutrophil percentage (%); RR=respiratory rate measured by chest impedance; HRxEDR=cross-correlation coefficient of heart rate and electrocardiogram-derived respiratory rate; Mg=magnesium; <RRI>=mean R-R interval; WBC=white blood cell count; Na=sodium; Hct=hematocrit; Cl=chloride; HRxSO2=the cross-correlation coefficient of heart rate and oxygen saturation; Alb=albumin; PCO2=partial pressure of carbon dioxide from arterial blood gas; sRRI=the standard deviation of R-R intervals; HRxRR=cross-correlation coefficient of heart rate measured by cardiac telemetry and respiratory rate measured by chest impedance; Ca=calcium; HRV=standard deviation of heart rate by cardiac telemetry (beats/min); DFA=detrended fluctuation analysis applied to R-R intervals; TP=total protein; BUN/Cr=BUN/creatinine; Plt=platelet count; Temp=temperature (C); RRV=standard deviation of respiratory rate by chest impedance (breaths/min); SBP=invasive systolic blood pressure (mmHg); MBP=invasive mean blood pressure (mmHg); SBP (cuff)=systolic blood pressure by cuff measurement; HCO3=bicarbonate; DBP (cuff)=diastolic blood pressure by cuff measurement; DBP=invasive diastolic blood pressure (mmHg); CO2=carbon dioxide; MAP=mean arterial pressure by cuff measurement; Gluc=glucose; BE=base excess from arterial blood gas; pH=pH from arterial blood gas).

Several of these variables had nonlinear associations with ICU hypoglycemia (e.g., white blood cell count, serum potassium, etc.), indicating that hypoglycemia risk increased at both the lowest and highest percentiles of these variables. Another notable finding was that serum anion gap demonstrated a strongly positive association with ICU hypoglycemia. We initially attributed this to diabetic ketoacidosis, but noted that lactic acid also demonstrated a strongly positive relationship with hypoglycemia risk. These results suggest that the positive relationship between ICU hypoglycemia risk and higher anion gap is likely indicative of severe or worsening illness due to factors (e.g., lactic acidosis, uremia, etc.) beyond diabetic ketoacidosis alone.

Multivariable logistic regression modeling identified a signature of 41 independent predictors that characterized impending ICU hypoglycemia. These features were, in decreasing strength of association: serum glucose, serum anion gap, body temperature, serum potassium, serum creatinine, prothrombin time, BUN/creatinine, serum carbon dioxide, the standard deviation of oxygen saturation by pulse oximetry (i.e., O2V), serum calcium, the standard deviation of respiratory rate by chest impedance (i.e., RRV), age, detrended fluctuation analysis applied to R-R intervals (i.e., DFA), the standard deviation of R-R intervals (i.e., sRRI), serum platelet count, serum hematocrit, clinician documented oxygen saturation (i.e., SpO2), mean R-R interval (i.e., <RRI>), serum phosphorous, diastolic blood pressure (cuff measurement), serum sodium, serum magnesium, white blood cell count, probability of atrial fibrillation (i.e., AF), respiratory rate measured by chest impedance (i.e., RR), serum blood urea nitrogen, male gender, cross-correlation coefficient of heart rate measured by cardiac telemetry and respiratory rate measured by chest impedance (i.e., HRxRR), electrocardiogram-derived respiratory rate using Kalman filtering (i.e., EDR(K)), pulse rate measured by pulse oximetry, coefficient of sample entropy of R-R interval (i.e., COSEn), oxygen saturation measured by pulse oximetry (i.e., SO2), local dynamics density of heart rate (i.e., LDd), the crosscorrelation coefficient of heart rate and oxygen saturation (i.e., HRxSO2), invasive diastolic blood pressure (measured by arterial line), cross-correlation coefficient of heart rate and electrocardiogram-derived respiratory rate (i.e., HRxEDR), standard deviation of heart rate by cardiac telemetry (i.e., HRV), respiratory rate measured by pulse oximetry (i.e., Resp), systolic blood pressure (cuff measurement), invasive systolic blood pressure (measured by arterial line), and cross-correlation coefficient of respiratory rate measured by chest impedance and oxygen saturation measured by pulse oximetry (i.e., RRxSO2).

### Model Validation and Performance

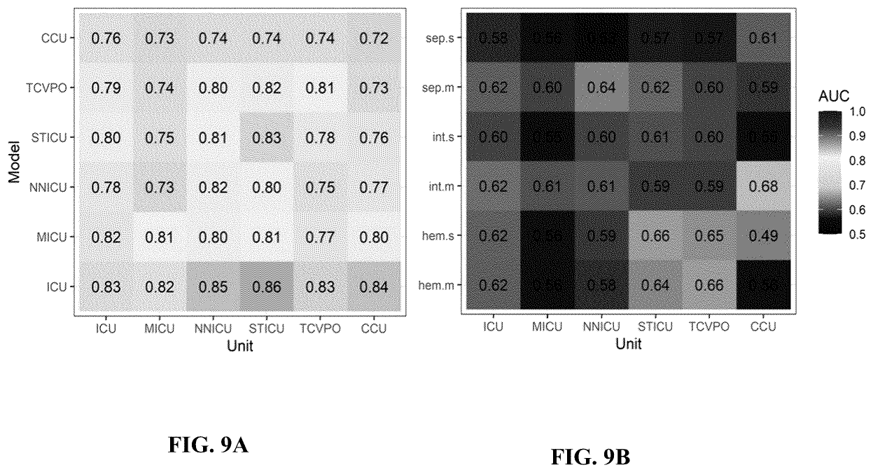

FIGS. 9A-9B show cross-validated AUROC for the ICU hypoglycemia model. FIGS. 9A-9B were generated in color but is presented here in grayscale. One skilled in the art would understand how to interpret the plot and equally understand what the results represent by using the grayscale. FIG. 9A shows area under the receiver operating characteristic curve (AUROC) values for the aggregate ICU hypoglycemia model and ICU-specific models. Values on the diagonals are cross-validated. FIG. 9B shows performance of prior models developed for prediction of ICU sepsis, intubation, and hemorrhage. (ICU=intensive care unit aggregate model; MICU=medical intensive care unit; NNICU=neuroscience intensive care unit; STICU=surgical-trauma intensive care unit; TCVPO=thoracic-cardiovascular postoperative intensive care unit; CCU=coronary care intensive care unit; sep.s=STICU sepsis model; sep.m=MICU sepsis model; int.s=STICU intubation model; int.m=MICU intubation model; hem.s=STICU hemorrhage model; hem.m=MICU hemorrhage model).

The cross-validated AUROC for our ICU hypoglycemia model was 0.83 (95% CI: 0.79-0.88) (see FIG. 9A), and the area under the precision-recall curve (AUPRC) was 0.094 (event rate=0.0045 and ratio=20.7). We also examined performance of prior models developed for prediction of ICU sepsis, intubation, and hemorrhage to determine their predictive capability for ICU hypoglycemia. These models all demonstrated poor predictive ability for ICU hypoglycemia (FIG. 9B). For example, a model for sepsis in the medical ICU had an AUROC of 0.62 for detection of ICU hypoglycemia. This suggests that our ICU hypoglycemia model is specific to hypoglycemia and not just worsening clinical status. We then sought to test the performance of our model in independent data sets not used for model development. Our model demonstrated good performance in MIMIC-III with an AUROC of 0.79 (95% CI: 0.77-0.81) and AUPRC of 0.09 (event rate=0.0082 and ratio=11.0). However, some limitations of the MIMIC-III dataset should be noted: (1) bedside monitoring blood pressures were significantly lower in some MIMIC ICUs; (2) bedside monitoring vital signs in MIMIC-III were often sampled every 1 minute instead of every 1 second, so the standard deviation differs from our UVA dataset; (3) time stamps for labs in MIMIC-III were blood draw time and not result time; and (4) medicine administration was not available to restrict analysis to only insulin-treated admissions.

### Model Calibration and Temporal Risk Association

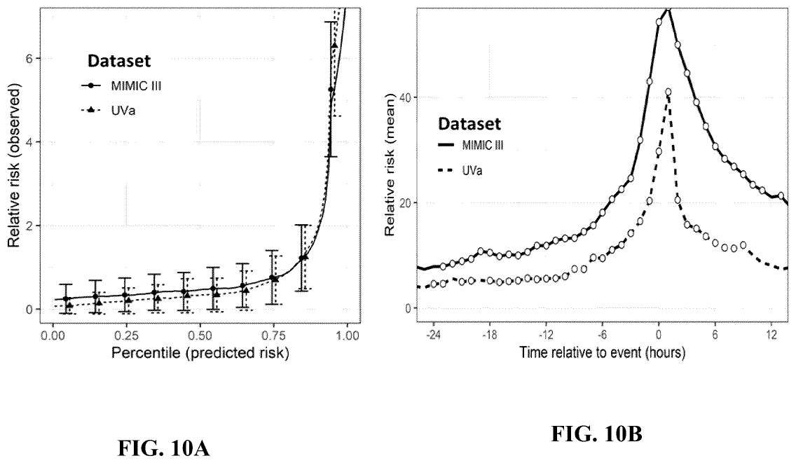

FIGS. 10A and 10B show plotted calibration curve for the aggregate ICU hypoglycemia model. FIG. 10A shows a calibration plot demonstrating goodness-of-fit for the ICU hypoglycemia model as a risk metric and classifier of impending ICU hypoglycemia in both the UVA and MIMIC-III datasets. The solid line represents hypoglycemia index values normalized by the average risk of 0.62% and plotted from lowest to highest. Dark circles represent proportion of ICU patients per decile with proven hypoglycemia in the next 24 hours. Error bars are based on the standard error of observed risk (proportion). (ICU=intensive care unit; UVA=University of Virginia). FIG. 10B shows average risk relative to hypoglycemic event as determined by the ICU hypoglycemia model in both the UVa and MIMIC III datasets. (UVa=University of Virginia).

The plotted calibration curve for the aggregate ICU hypoglycemia model is shown in FIG. 10A. The model demonstrated reasonable calibration within both the UVA and MIMIC-III datasets, with predicted risk rising as relative risk increased. Notably, in both datasets, patients with the lowest 80% of predicted risk had less than average observed risk. FIG. 10B demonstrates average risk in relation to timing of hypoglycemic events. The model identified rising hypoglycemia risk ˜4-6 hours prior to the hypoglycemic event in both the UVA and MIMIC-III datasets, reflecting a rising degree of physiological and biochemical abnormality in the hours prior to clinical recognition of hypoglycemia.

### DISCUSSION

With a rich ICU dataset collected from the EHR of a large university hospital, we utilized a “Big Data” analytic approach and applied multivariable logistic regression to describe signatures of ICU hypoglycemia from readily-available physiological and biochemical data. The comprehensive signature was composed of 41 different variables and demonstrated good discriminatory capability and reasonable calibration (28). To our knowledge, this is the first study that incorporates hemodynamic and electrophysiological bedside monitoring data to provide a comprehensive and quantitative predictive model of the clinical pathophysiology of ICU hypoglycemia.

The features of our aggregate model described a pathophysiologic signature of impending ICU hypoglycemia that was consistent across different adult ICUs. This signature was independent of recent blood glucose trends and had general similarities to other illnesses (e.g., hypoglycemia, hypothermia, increasing anion gap, hypocalcemia, etc. are metabolic and hemodynamic derangements frequently seen in critically ill states (11, 29-32)), but was sufficiently different to warrant its own model. The model identified rising hypoglycemia risk ˜4-6 hours prior to the hypoglycemic event in both the UVA and MIMIC-III datasets, suggesting that there is a reasonable timeframe for early intervention prior to occurrence of a hypoglycemic event.

Machine learning has been increasingly utilized to develop predictive models for inpatient hypoglycemia (15, 17, 33-36). Only one report thus far focused on predicting solely ICU hypoglycemia, and this study utilized classification tree learning for model development (17). Several other models utilized logistic or multivariable regression techniques for prediction of inpatient hypoglycemia, but these studies examined only inpatients with a diagnosis of diabetes mellitus (15, 35), only inpatients who experienced severe hypoglycemia (33, 34), and only non-critically ill inpatients (36, 37). Ruan et al. recently used average values from the entire admission (based on data available after the admission ended) to compare the ability of advanced machine learning and logistic regression models to retrospectively estimate the risk of hypoglycemia in inpatients with diabetes (15). The model we present, by contrast, is appropriate for risk prediction at any point during the ICU stay based only on data available at that time. Similar to the present study, Mathioudakis et al. recently developed and validated a machine learning model to predict near-term risk of iatrogenic hypoglycemia in hospitalized patients (37). Their model, however, was trained on and specifically developed for non-ICU admissions and did not exclude blood glucose as a predictor. Our results show that an aggregate ICU hypoglycemia model (including blood glucose, biochemical, and electrophysiological monitoring data) demonstrated significantly higher AUROC values at every detection window when compared to models based on blood glucose alone and hemodynamic/electrophysiological monitoring data alone.

The findings of the present study advance the work of other groups and demonstrate that hemodynamic and electrophysiological data augment biochemical data to improve predictive models for ICU hypoglycemia.

Appropriate glycemic control is a necessary component of quality-driven inpatient healthcare. In critically-ill inpatients, intensive glycemic control reduces hyperglycemia but often leads to subsequent hypoglycemia (11). The NICE-SUGAR trial found that intensive insulin therapy increased 90-day mortality compared with conventional treatment in ICU patients (38). In that trial, the incidence of severe hypoglycemia was significantly higher with intensive insulin therapy compared to conventional treatment.

Other work has shown that even mild hypoglycemia is strongly associated with increased ICU length-of-stay (13). Our study found similar associations between ICU hypoglycemia, mortality, and length-of-stay. We cannot prove causality for these associations, and it may be that hypoglycemia itself is a clinical sign of worsening or severe illness. However, one recent study found that reducing the incidence of inpatient hypoglycemia concomitantly reduced inpatient and 30-day mortality rates (39). Further trials are needed to determine if direct reduction of ICU hypoglycemia improves clinical outcomes. The model has been utilized “in the background” of real-time ICU admissions to determine if it prospectively predicts hypoglycemia and determine what clinical events might be prevented with earlier intervention. The model can be incorporated into a clinical decision support system.

Our study has several strengths that should be noted, including the large dataset used for model development. Another strength is the model's ability to immediately quantify the change in hypoglycemia risk from small changes in any of its physiologic variables and produce a new and continuously-updated estimate of ICU hypoglycemia risk in a given patient. The use of variables that are easily-accessible from EHR and bedside monitoring data allow for integration into a clinical decision support system that suggests appropriate interventions based on individual risk levels, ultimately providing a personalized approach to ICU hypoglycemia.

There are also limitations of this study that warrant discussion. First, our model was generated using single-center, retrospective, observational data. Second, our EHR dataset does not quantify status of hypoglycemia awareness, continuous blood glucose monitoring values, or blood glucose self-monitoring values prior to admission. These data may be an important factor in developing inpatient hypoglycemia, though others have pointed out that such data may not be directly applicable to critically-ill patients (15).

Finally, there was no information readily-available in the EHR regarding oral or enteral nutrition intake status. The lack of such data does not alter our model's ability to predict ICU hypoglycemia, but its inclusion would likely be informative.

### CONCLUSIONS

In summary, we used data from a large number of critically-ill adult patients to test the hypothesis that routine monitoring variables could describe a detailed and distinct pathophysiologic phenotype of ICU hypoglycemia. This physiologic signature could provide a basis for future predictive modeling by improving recognition of impending ICU hypoglycemia, informing the design of earlier interventions and measuring their effectiveness, and identifying opportunities for the development of novel therapeutics.

An aspect of an embodiment of the present invention provides a system, method and computer readable medium for, among other things, one or more of the following: a) providing a predictive model of impending intensive care unit hypoglycemia; b) providing a predictive model for ICU hypoglycemia that may provide a basis for future real-time predictive modeling that will improve recognition of impending hypoglycemia and direct earlier administration of preventive therapy in ICU patients; c) the ability to incorporate hemodynamic and electrophysiological bedside monitoring data to provide a comprehensive and quantitative predictive model of the clinical pathophysiology of ICU hypoglycemia; d) providing a predictive model that identifies rising hypoglycemia risk in a specified period of time (e.g., ˜4-6 hours) prior to the hypoglycemic event, suggesting that there is a reasonable timeframe for early intervention prior to occurrence of a hypoglycemic event; e) providing a predictive model that offers clinical impact; f) providing a model that prospectively predicts hypoglycemia and determine what clinical events might be prevented with earlier intervention; or g) providing a predictive model that yields routine monitoring variables that could describe, but not limited thereto, a detailed and distinct pathophysiologic phenotype of ICU hypoglycemia.

An aspect of an embodiment of the present invention provides a system, method and computer readable medium for providing, among other things, pathophysiologic signature of impending ICU hypoglycemia in bedside monitoring and electronic health record data.

An aspect of an embodiment of the present invention provides a system, method and

computer readable medium for providing, among other things, a predictive model for determining the Pathophysiologic signature of hypoglycemia.

Although example embodiments of the present disclosure are explained in some instances in detail herein, it is to be understood that other embodiments are contemplated.

Accordingly, it is not intended that the present disclosure be limited in its scope to the details of construction and arrangement of components set forth in the following description or illustrated in the drawings. The present disclosure is capable of other embodiments and of being practiced or carried out in various ways.

It will be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

It will be appreciated that the device and related components discussed herein may take on all shapes along the entire continual geometric spectrum of manipulation of x, y and z planes to provide and meet the environmental, anatomical, and structural demands and operational requirements. Moreover, locations and alignments of the various components may vary as desired or required.

It will be appreciated that various sizes, dimensions, contours, rigidity, shapes, flexibility and materials of any of the components or portions of components in the various embodiments discussed throughout may be varied and utilized as desired or required.

It will be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

It will be appreciated that as discussed herein, a subject may be a human or any animal. It will be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It will be appreciated that the subject may be any applicable human patient, for example.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Additional descriptions of aspects of the present disclosure will now be provided with reference to the accompanying drawings. The drawings form a part hereof and show, by way of illustration, specific embodiments or examples.

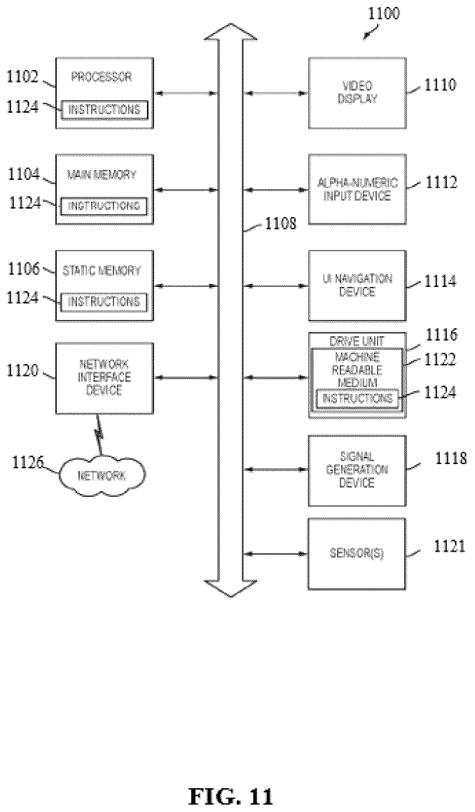

FIG. 11 is a block diagram illustrating an example of a machine upon which one or more aspects of embodiments of the present invention can be implemented.

Referring to FIG. 11, an aspect of an embodiment of the present invention includes, but not limited thereto, a system, method, and computer readable medium that provides: a model for identifying and predicting hypoglycemia risk, which illustrates a block diagram of an example machine 1100 upon which one or more embodiments (e.g., discussed methodologies) can be implemented (e.g., run).

Examples of machine 1100 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium (e.g., non-transitory, non-volatile memory) or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time.

For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs)).

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a standalone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 1100) and software architectures that can be deployed in example embodiments.

In an example, the machine 1100 can operate as a standalone device or the machine 1100 can be connected (e.g., networked) to other machines.

In a networked deployment, the machine 1100 can operate in the capacity of either a server or a client machine in server-client network environments. In an example, machine 1100 can act as a peer machine in peer-to-peer (or other distributed) network environments. The machine 1100 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 1100. Further, while only a single machine 1100 is illustrated, the term “machine” shall also be taken to include any collection of machines that individually or jointly execute a set (or multiple sets) of instructions to perform any one or more of the methodologies discussed herein.

Example machine (e.g., computer system) 1100 can include a processor 1102 (e.g., a central processing unit (CPU), a graphics processing unit (GPU) or both), a main memory 1104 and a static memory 1106, some or all of which can communicate with each other via a bus 1108.

The machine 1100 can further include a display unit 1110, an alphanumeric input device 1112 (e.g., a keyboard), and a user interface (UI) navigation device 1114 (e.g., a mouse). In an example, the display unit 1110, input device 1117 and UI navigation device 1114 can be a touch screen display. The machine 1100 can additionally include a storage device (e.g., drive unit) 1116, a signal generation device 1118 (e.g., a speaker), a network interface device 1120, and one or more sensors 1121, such as a global positioning system (GPS) sensor, compass, accelerometer, or other sensor.

The storage device 1116 can include a machine readable medium 1122 on which is stored one or more sets of data structures or instructions 1124 (e.g., software) embodying or utilized by any one or more of the methodologies or functions described herein. The instructions 1124 can also reside, completely or at least partially, within the main memory 1104, within static memory 1106, or within the processor 1102 during execution thereof by the machine 1100. In an example, one or any combination of the processor 1102, the main memory 1104, the static memory 1106, or the storage device 1116 can constitute machine readable media. While the machine readable medium 1122 is illustrated as a single medium, the term “machine readable medium” can include a single medium or multiple media (e.g., a centralized or distributed database, and/or associated caches and servers) that configured to store the one or more instructions 1124. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 1124 can further be transmitted or received over a communications network 1126 using a transmission medium via the network interface device 1120 utilizing any one of a number of transfer protocols (e.g., frame relay, IP, TCP, UDP, HTTP, etc.).

Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the disclosure (and claims) including all modifications and equivalents.

Still other embodiments will become readily apparent to those skilled in this art from reading the above-recited detailed description and drawings of certain exemplary embodiments. It will be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

The following references are incorporated herein by reference in their entireties.
1. Griffing K L: Hypoglycemia Prevention in Hospital Patients: A Quality Improvement Project to Prevent Severe and Recurrent Hypoglycemia. Clin Diabetes 2016; 34(4): 193-199.
2. Corsino L, Dhatariya K, Umpierrez G: Management of Diabetes and Hyperglycemia in Hospitalized Patients. In: Endotext Feingold K R, Anawalt B, Boyce A, Chrousos G, et al (Ed).
3. Cryer P E: Death during intensive glycemic therapy of diabetes: mechanisms and implications. Am J Med 2011; 124(11):993-996.
4. Desouza C, Salazar H, Cheong B, Murgo J, et al: Association of hypoglycemia and cardiac ischemia: a study based on continuous monitoring. Diabetes Care 2003; 26(5): 1485-1489.
5. Frier B M, Schernthaner G, Heller S R: Hypoglycemia and cardiovascular risks. Diabetes Care 2011; 34 Suppl 2:S132-137.
6. Egi M, Bellomo R, Stachowski E, French C J, et al: Hypoglycemia and outcome in critically ill patients. Mayo Clin Proc 2010; 85(3): 217-224.
7. Akirov A, Grossman A, Shochat T, Shimon I: Mortality Among Hospitalized Patients With Hypoglycemia: Insulin Related and Noninsulin Related. J Clin Endocrinol Metab 2017; 102(2): 1116-424.
8. Pratley R: At a Cost of $10,405 Per Patient Stay, Hypoglycemia in The Hospital Cannot Be Ignored. Available at: https://glytecsystems.com/news/at-a-cost-of-10-405-per-patient-stayhypoglycemia-in-the-hospital-cannot-be-ignored/. Accessed 01/20/2021.
9. Cook C B, Kongable G L, Potter D J, Abad V J, et al: Inpatient glucose control: a glycemic survey of 126 U.S. hospitals. J Hosp Med 2009; 4(9): E7-E14.
10. Hulkower R D, Pollack R M, Zonszein J: Understanding hypoglycemia in hospitalized patients. Diabetes Manag (Lond) 2014; 4(2):165-176
11. Investigators N-SS, Finfer S, Liu B, Chittock D R, et al: Hypoglycemia and risk of death in critically ill patients. N Engl J Med 2012; 367(12): 1108-1118.
12. Krinsley J S, Schultz M J, Spronk P E, Harmsen R E, et al: Mild hypoglycemia is independently associated with increased mortality in the critically ill. Crit Care 2011; 15(4):R173.
13. Krinsley J, Schultz M J, Spronk P E, van Braam Houckgeest F, et al: Mild hypoglycemia is strongly associated with increased intensive care unit length of stay. Annals of Intensive Care 2011; 1(1).
14. Davis J P, Wessells D A, Moorman J R: Coronavirus Disease 2019 Calls for Predictive Analytics Monitoring-A New Kind of Illness Scoring System. Crit Care Explor 2020; 2(12): e0294.
15. Ruan Y, Bellot A, Moysova Z, Tan G D, et al: Predicting the Risk of Inpatient Hypoglycemia With Machine Learning Using Electronic Health Records. Diabetes Care 2020; 43(7): 1504-1511.
16. Tsujimoto T, Yamamoto-Honda R: Clinical conditions during severe hypoglycemia in patients with diabetes. Diabetology International 2014; 5(2):81-83.
17. Zhang Y: Predicting occurrences of acute hypoglycemia during insulin therapy in the intensive care unit. Annu Int Conf IEEE Eng Med Biol Soc 2008; 2008:3297-3300.
18. Collins G S, Reitsma J B, Altman D G, Moons K G: Transparent Reporting of a multivariable prediction model for Individual Prognosis or Diagnosis (TRIPOD): the TRIPOD statement. Ann Intern Med 2015; 162(1):55-63.
19. Leisman D E, Harhay M O, Lederer D J, Abramson M, et al: Development and Reporting of Prediction Models: Guidance for Authors From Editors of Respiratory, Sleep, and Critical Care Journals. Crit Care Med 2020; 48(5):623-633.
20. Moons K G, Altman D G, Reitsma J B, Ioannidis J P, et al: Transparent Reporting of a multivariable prediction model for Individual Prognosis or Diagnosis (TRIPOD): explanation and elaboration. Ann Intern Med 2015; 162(1): W1-73.
21. Harrell F E: Regression modeling strategies: with applications to linear models, logistic regression, and survival analysis. New York, Springer, 2001.
22 Moss T J, Lake D E, Calland J F, Enfield K B, et al: Signatures of Subacute Potentially Catastrophic Illness in the ICU: Model Development and Validation. Crit Care Med 2016; 44(9):1639-1648.
23. Hoerl A E, Kennard R W: Ridge Regression: Biased Estimation for Nonorthogonal Problems. Technometrics 1970; 12(1):55-67.
24. Zimmet A N, Clark M T, Gadrey S M, Bell T D, et al: Pathophysiologic Signatures of Bloodstream Infection in Critically Ill Adults. Crit Care Explor 2020; 2(10):e0191.
25. Hurvich C, Tsai C: Regression and time series model selection in small samples. Biometrika 1989; 76:297-307.
26. Hawkins D M, Basak S C, Mills D: Assessing model fit by cross-validation. J Chem Inf Comput Sci 2003; 43(2):579-586.
27. Johnson A E, Pollard T J, Shen L, Lehman L W, et al: MIMIC-III, a freely accessible critical care database. Sci Data 2016; 3:160035.
28. Fan J, Upadhye S, Worster A: Understanding receiver operating characteristic (ROC) curves. CJEM 2006; 8(1):19-20.
29. McCowen K C, Malhotra A, Bistrian B R: Stress-induced hyperglycemia. Crit Care Clin 2001; 17(1): 107-124.
30. Zivin J R, Gooley T, Zager R A, Ryan M J: Hypocalcemia: a pervasive metabolic abnormality in the critically ill. Am J Kidney Dis 2001; 37(4):689-698.
31. Al-Jaghbeer M, Kellum J A: Acid-base disturbances in intensive care patients: etiology, pathophysiology and treatment. Nephrol Dial Transplant 2015; 30(7):1104-1111.
32. Kushimoto S, Yamanouchi S, Endo T, Sato T, et al: Body temperature abnormalities in nonneurological critically ill patients: a review of the literature. J Intensive Care 2014; 2(1): 14.
33. Kilpatrick C R, Elliott M B, Pratt E, Schafers S J, et al: Prevention of inpatient hypoglycemia with a real-time informatics alert. J Hosp Med 2014; 9(10):621-626.
34. Elliott M B, Schafers S J, McGill J B, Tobin G S: Prediction and prevention of treatment-related inpatient hypoglycemia. J Diabetes Sci Technol 2012; 6(2):302-309.
35. Stuart K, Adderley N J, Marshall T, Rayman G, et al: Predicting inpatient hypoglycaemia inhospitalized patients with diabetes: a retrospective analysis of 9584 admissions with diabetes. Diabet Med 2017; 34(10): 1385-1391.
36. Mathioudakis N N, Everett E, Routh S, Pronovost P J, et al: Development and validation of a prediction model for insulin-associated hypoglycemia in non-critically ill hospitalized adults. BMJ Open Diabetes Res Care 2018; 6(1): e000499.
37. Mathioudakis N N, Abusamaan M S, Shakarchi A F, Sokolinsky S, et al: Development andValidation of a Machine Learning Model to Predict Near-Term Risk of Iatrogenic Hypoglycemia in Hospitalized Patients. JAMA Netw Open 2021; 4(1):e2030913.
38. Investigators N-SS, Finfer S, Chittock D R, Su S Y, et al: Intensive versus conventional glucose control in critically ill patients. N Engl J Med 2009; 360(13): 1283-1297.
39. Kana Kadayakkara D, Balasubramanian P, Araque K, Davis K, et al: Multidisciplinary strategies to treat severe hypoglycemia in hospitalized patients with diabetes mellitus reduce inpatient mortality rate: Experience from an academic community hospital. PLoS One 2019; 14(8):e0220956.
40. John P. Davis, MD, Dustin A. Wessells, and J. Randall Moorman, MD: Coronavirus Disease 2019 Calls for Predictive Analytics Monitoring-A New Kind of Illness Scoring System, Critical Care Explorations, 2020.
41. Oliver Monfredi, Jessica Keim-Malpass, and J Randall Moorman: Continuous cardiorespiratory monitoring is a dominant source of predictive signal in machine learning for risk stratification and clinical decision support, Physiological Measurement, 2021.
42. The Satires of Juvenal, Persius, Sulpicia, and Lucilius. (2015). Project Gutenberg.
43. How IBM Watson Overpromised and Underdelivered on AI Health Care-IEEE Spectrum.
44. https://spectrum.ieee.org/how-ibm-watson-overpromised-and-underdelivered-on-ai-health-care.
45. Wong, A. et al. External validation of a widely implemented proprietary sepsis prediction model in hospitalized patients. JAMA Intern. Med. 181, 1065-1070 (2021).
46. Wong, A. et al. Quantification of Sepsis Model Alerts in 24 US Hospitals Before and During the COVID-19 Pandemic. JAMA Netw. Open 4, e2135286 (2021).
47. Beaulieu-Jones, B. K. et al. Machine learning for patient risk stratification: standing on, or looking over, the shoulders of clinicians? npj Digital Med. 4, 62 (2021).
48. Moorman, J. R. et al. Mortality reduction by heart rate characteristic monitoring in very low birth weight neonates: a randomized trial. J. Pediatr. 159, 900-6.e1 (2011).
49. Griffin, M. P. & Moorman, J. R. Toward the early diagnosis of neonatal sepsis and sepsis-like illness using novel heart rate analysis. Pediatrics 107, 97-104 (2001).
50. Richman, J. S. & Moorman, J. R. Physiological time-series analysis using approximate entropy and sample entropy. Am. J. Physiol. Heart Circ. Physiol. 278, H2039-49 (2000).
51. Lake, D. E., Richman, J. S., Griffin, M. P. & Moorman, J. R. Sample entropy analysis of neonatal heart rate variability. Am. J. Physiol. Regul. Integr. Comp. Physiol. 283, R789-97 (2002).
52. Kovatchev, B. P. et al. Sample asymmetry analysis of heart rate characteristics with application to neonatal sepsis and systemic inflammatory response syndrome. Pediatr. Res. 54, 892-898 (2003).
53. Moorman, J. R. et al. Cardiovascular oscillations at the bedside: early diagnosis of neonatal sepsis using heart rate characteristics monitoring. Physiol. Meas. 32, 1821-1832 (2011).
54. Griffin, M. P. et al. Abnormal heart rate characteristics preceding neonatal sepsis and sepsis-like illness. Pediatr. Res. 53, 920-926 (2003).
55. Swanson, J. R. et al. Neonatal intensive care unit length of stay reduction by heart rate characteristics monitoring. J. Pediatr. 198, 162-167 (2018).
56. King, W. E., Carlo, W. A., O'Shea, T. M., Schelonka, R. L. & HRC neurodevelopmental follow-up investigators. Heart rate characteristics monitoring and reduction in mortality or neurodevelopmental impairment in extremely low birthweight infants with sepsis. Early Hum. Dev. 159, 105419 (2021).
57. Schelonka, R. L. et al. Mortality and neurodevelopmental outcomes in the heart rate characteristics monitoring randomized controlled trial. J. Pediatr. 219, 48-53 (2020).
58. Fairchild, K. D. et al. Septicemia mortality reduction in neonates in a heart rate characteristics monitoring trial. Pediatr. Res. 74, 570-575 (2013).
59. Liu, V. X. et al. The timing of early antibiotics and hospital mortality in sepsis. Am. J. Respir. Crit. Care Med. 196, 856-863 (2017).
60. Stoll, B. J. et al. Late-onset sepsis in very low birth weight neonates: the experience of the NICHD Neonatal Research Network. Pediatrics 110, 285-291 (2002).
61. Moss, T. J. et al. Signatures of subacute potentially catastrophic illness in the ICU: model development and validation. Crit. Care Med. 44, 1639-1648 (2016).
62. Moss, T. J. et al. Cardiorespiratory dynamics measured from continuous ECG monitoring improves detection of deterioration in acute care patients: A retrospective cohort study. PLoS ONE 12, e0181448 (2017).
63. Spaeder, M. C. et al. Predictive analytics in the pediatric intensive care unit for early identification of sepsis: capturing the context of age. Pediatr. Res. 86, 655-661 (2019).
64. Politano, A. D. et al. Predicting the need for urgent intubation in a surgical/trauma intensive care unit. Surgery 154, 1110-1116 (2013).
65. Callcut, R. A. et al. External validation of a novel signature of illness in continuous cardiorespiratory monitoring to detect early respiratory deterioration of ICU patients. Physiol. Meas. 42, (2021).
66. De Pasquale, M. et al. Hemorrhage prediction models in surgical intensive care: bedside monitoring data adds information to lab values. IEEE J. Biomed. Health Inform. 21, 1703-1710 (2017).
67 Moss, T. J., Clark, M. T., Lake, D. E., Moorman, J. R. & Calland, J. F. Heart rate dynamics preceding hemorrhage in the intensive care unit. J. Electrocardiol. 48, 1075-1080 (2015).
68. Horton, W. B., Barros, A. J., Andris, R. T., Clark, M. T. & Moorman, J. R. Pathophysiologic signature of impending ICU hypoglycemia in bedside monitoring and electronic health record data: model development and external validation. Crit Care Med In press, (2021).
69. Blackwell, J. N. et al. Early detection of in-patient deterioration: one prediction model does not fit all. Critical Care Explorations (2020).
70. Rothman, M. J., Rothman, S. I. & Beals, J. Development and validation of a continuous measure of patient condition using the Electronic Medical Record. J. Biomed. Inform. 46, 837-848 (2013).
71. Burnard, E. D. Changes in heart size in the dyspnoeic newborn infant. British Medical Journal 1, 1495-1500 (1959).
72. Rudolph, A. J., Vallbona, C. & Desmond, M. M. Cardiodynamic studies in the newborn. III. Heart rate patterns in infants with idiopathic respiratory distress syndrome. Pediatrics 36, 551-559 (1965).
73. Cabal, L. A., Siassi, B., Zanini, B., Hodgman, J. E. & Hon, E. E. Factors affecting heart rate variability in preterm infants. Pediatrics 65, 50-56 (1980).
74. Hon, E. H. The electronic evaluation of the fetal heart rate; preliminary report. Am J Obstet Gynecol 75, 1215-1230 (1958).
75. Hon, E. H. & Lee, S. T. Electronic evaluations of the fetal heart rate patterns preceding fetal death: further observations. American Journal of Obstetrics and Gynecology 814-826 (1965).
76. Stone, M. L. et al. Abnormal heart rate characteristics before clinical diagnosis of necrotizing enterocolitis. J. Perinatol. 33, 847-850 (2013).
77 Dall'Ora, C. et al. Nurses' 12-hour shifts and missed or delayed vital signs observations on hospital wards: retrospective observational study. BMJ Open 9, e024778 (2019).
78. Karemaker, J. M. An introduction into autonomic nervous function. Physiol. Meas. 38, R89-R118 (2017).
79. Monfredi, O., Keim-Malpass, J. & Moorman, J. R. Continuous cardiorespiratory monitoring is a dominant source of predictive signal in machine learning for risk stratification and clinical decision support. Physiol. Meas. 42, (2021).
80. Davis, J. P., Wessells, D. A. & Moorman, J. R. Coronavirus Disease 2019 Calls for Predictive Analytics Monitoring-A New Kind of Illness Scoring System. Crit. Care Explor. 2, e0294 (2020).
81. Saria, S., Rajani, A. K., Gould, J., Koller, D. & Penn, A. A. Integration of early physiological responses predicts later illness severity in preterm infants. Sci. Transl. Med. 2, 48ra65 (2010).
82. Tarassenko, L., Hann, A. & Young, D. Integrated monitoring and analysis for early warning of patient deterioration. Br. J. Anaesth. 97, 64-68 (2006).
83. Ruminski, C. M. et al. Impact of predictive analytics based on continuous cardiorespiratory monitoring in a surgical and trauma intensive care unit. J. Clin. Monit. Comput. 33, 703-711 (2019).
84. Griffin, M. P., Lake, D. E. & Moorman, J. R. Heart rate characteristics and laboratory tests in neonatal sepsis. Pediatrics 115, 937-941 (2005).
85. Griffin, M. P., Lake, D. E., O'Shea, T. M. & Moorman, J. R. Heart rate characteristics and clinical signs in neonatal sepsis. Pediatr. Res. 61, 222-227 (2007).
86. Henry, K. E., Hager, D. N., Pronovost, P. J. & Saria, S. A targeted real-time early warning score (TREWScore) for septic shock. Sci. Transl. Med. 7, 299ra122 (2015).
87. Xiao, Y., Griffin, M. P., Lake, D. E. & Moorman, J. R. Nearest-neighbor and logistic regression analyses of clinical and heart rate characteristics in the early diagnosis of neonatal sepsis. Med. Decis. Making 30, 258-266 (2010).
88. Griffin, M. P., Scollan, D. F. & Moorman, J. R. The dynamic range of neonatal heart rate variability.
89. J. Cardiovasc. Electrophysiol. 5, 112-124 (1994).
90 Aghili, A. A., Rizwan-uddin, Griffin, M. P. & Moorman, J. R. Scaling and ordering of neonatal heart rate variability. Phys. Rev. Lett. 74, 1254-1257 (1995).
91. Nelson, J. C., Rizwan-uddin, Griffin, M. P. & Moorman, J. R. Probing the order within neonatal heart rate variability. Pediatr. Res. 43, 823-831 (1998).
92. Chang, K. L., Monahan, K. J., Griffin, M. P., Lake, D. & Moorman, J. R. Comparison and clinical application of frequency domain methods in analysis of neonatal heart rate time series. Ann. Biomed. Eng. 29, 764-774 (2001).
93. Lee, H. et al. A new algorithm for detecting central apnea in neonates. Physiol. Meas. 33, 1-17 (2012).
94. Flower, A. A., Moorman, J. R., Lake, D. E. & Delos, J. B. Periodic heart rate decelerations in premature infants. Exp Biol Med (Maywood) 235, 531-538 (2010).
95. Mohr, M. A. et al. Quantification of periodic breathing in premature infants. Physiol. Meas. 36, 1415-1427 (2015).
96. Clark, M. T. et al. Breath-by-breath analysis of cardiorespiratory interaction for quantifying developmental maturity in premature infants. J. Appl. Physiol. 112, 859-867 (2012).
97. Cao, H., Lake, D. E., Griffin, M. P. & Moorman, J. R. Increased nonstationarity of neonatal heart rate before the clinical diagnosis of sepsis. Ann. Biomed. Eng. 32, 233-244 (2004).
98. Fulcher, B. D., Little, M. A. & Jones, N. S. Highly comparative time-series analysis: the empirical structure of time series and their methods. J. R. Soc. Interface 10, 20130048 (2013).
99. Fulcher, B. D. & Jones, N. S. hctsa: A Computational Framework for Automated Time-Series Phenotyping Using Massive Feature Extraction. Cell Syst. 5, 527-531.e3 (2017).
100. Niestroy, J. C. et al. Discovery of signatures of fatal neonatal illness in vital signs using highly comparative time-series analysis. npj Digital Med. 5, 6 (2022).
101. Apgar, V. A proposal for a new method of evaluation of the newborn infant. Curr. Res. Anesth. Analg. 32, 260-267 (1953).
102. Richardson, D. K., Gray, J. E., McCormick, M. C., Workman, K. & Goldmann, D. A. Score for Neonatal Acute Physiology: a physiologic severity index for neonatal intensive care. Pediatrics 91, 617-623 (1993).
103. Vincent, J. L. et al. The SOFA (Sepsis-related Organ Failure Assessment) score to describe organ dysfunction/failure. On behalf of the Working Group on Sepsis-Related Problems of the European Society of Intensive Care Medicine. Intensive Care Med. 22, 707-710 (1996).
104. Wynn, J. L. & Polin, R. A. A neonatal sequential organ failure assessment score predicts mortality to late-onset sepsis in preterm very low birth weight infants. Pediatr. Res. 88, 85-90 (2020).
105. Collins, G. S., Ogundimu, E. O., Cook, J. A., Manach, Y. L. & Altman, D. G. Quantifying the impact of different approaches for handling continuous predictors on the performance of a prognostic model. Stat. Med. 35, 4124-4135 (2016).
106. Christodoulou, E. et al. A systematic review shows no performance benefit of machine learning over logistic regression for clinical prediction models. J. Clin. Epidemiol. 110, 12-22 (2019).
107. Johnson, K. W. et al. Artificial intelligence in cardiology. J. Am. Coll. Cardiol. 71, 2668-2679 (2018).
108. Khera, R. et al. Use of machine learning models to predict death after acute myocardial infarction. JAMA Cardiol. (2021) doi:10.1001/jamacardio.2021.0122.
109. Engelhard, M. M., Navar, A. M. & Pencina, M. J. Incremental Benefits of Machine Learning-When Do We Need a Better Mousetrap? JAMA Cardiol. (2021) doi:10.1001/jamacardio.2021.0139.
110. Mlodzinski, E., Stone, D. J. & Celi, L. A. Machine learning for pulmonary and critical care medicine: A narrative review. Pulm. Ther. (2020) doi:10.1007/s41030-020-00110-z.
111. Celi, L. A., Mark, R. G., Stone, D. J. & Montgomery, R. A. “Big data” in the intensive care unit. Closing the data loop. Am. J. Respir. Crit. Care Med. 187, 1157-1160 (2013).
112. Ghassemi, M., Celi, L. A. & Stone, D. J. State of the art review: the data revolution in critical care Crit. Care 19, 118 (2015).
113. Rudin, C. Stop explaining black box machine learning models for high stakes decisions and use interpretable models instead. Nat. Mach. Intell. 1, 206-215 (2019).
114. Harrell, F. E., Lee, K. L., Califf, R. M., Pryor, D. B. & Rosati, R. A. Regression modelling strategies for improved prognostic prediction. Stat. Med. 3, 143-152 (1984).
115. Rajkomar, A., Dean, J. & Kohane, I. Machine learning in medicine. N Engl J Med 380, 1347-58 (2019).
116. Rajkomar, A. et al. Scalable and accurate deep learning with electronic health records. npj Digital Med. 1, 18 (2018).
117. van Mourik, M. S. M., van Duijn, P. J., Moons, K. G. M., Bonten, M. J. M. & Lee, G. M. Accuracy of administrative data for surveillance of healthcare-associated infections: a systematic review. BMJ Open 5, e008424 (2015).
118. Rhee, C. et al. Incidence and Trends of Sepsis in US Hospitals Using Clinical vs Claims Data, 2009-2014. JAMA 318, 1241-1249 (2017).
119. Ghassemi, M., Oakden-Rayner, L. & Beam, A. L. The false hope of current approaches to explainable artificial intelligence in health care. Lancet Digit. Health 3, e745-e750 (2021).
120. Lake, D. E., Fairchild, K. D. & Moorman, J. R. Complex signals bioinformatics: evaluation of heart rate characteristics monitoring as a novel risk marker for neonatal sepsis. J. Clin. Monit. Comput. 28, 329-339 (2014).
121. Zimmet, A. M., Sullivan, B. A., Moorman, J. R., Lake, D. E. & Ratcliffe, S. J. Trajectories of the heart rate characteristics index, a physiomarker of sepsis in premature infants, predict Neonatal.
122. ICU mortality. JRSM Cardiovasc. Dis. 9, 2048004020945142 (2020).
123. Keim-Malpass, J. et al. Advancing Continuous Predictive Analytics Monitoring: Moving from Implementation to Clinical Action in a Learning Health System. Crit. Care Nurs. Clin. North Am. 30, 273-287 (2018).
124. Kitzmiller, R. R. et al. Diffusing an innovation: clinician perceptions of continuous predictive analytics monitoring in intensive care. Appl. Clin. Inform. 10, 295-306 (2019).
125. Escobar, G. J. et al. Automated Identification of Adults at Risk for In-Hospital Clinical Deterioration. N. Engl. J. Med. 383, 1951-1960 (2020).
126. Fanaroff, A. C. et al. Randomized trials versus common sense and clinical observation: JACC review topic of the week. J. Am. Coll. Cardiol. 76, 580-589 (2020).
127. Angus, D. C. Fusing Randomized Trials With Big Data: The Key to Self-learning Health Care Systems? JAMA 314, 767-768 (2015).
128. Leisman, D. E. Rare events in the ICU: an emerging challenge in classification and prediction. Crit. Care Med. 46, 418-424 (2018).
129. Coggins, S. A. et al. Heart rate characteristic index monitoring for bloodstream infection in an NICU: a 3-year experience. Arch. Dis. Child. Fetal Neonatal Ed. 101, F329-32 (2016).
130. Moorman, L. P. Principles for Real-World Implementation of Bedside Predictive Analytics Monitoring. Appl. Clin. Inform. 12, 888-896 (2021).
131. Kompa, B., Snoek, J. & Beam, A. L. Second opinion needed: communicating uncertainty in medical machine learning. npj Digital Med. 4, 4 (2021).
132. U.S. Utility patent application Ser. No. 15/973,024, entitled “Analysis of Cardiac Rhythm Using RR Interval Characterization”, filed May 7, 2018.
133. U.S. Pat. No. 10,582,873, issued Mar. 10, 2020.
134. U.S. Utility patent application Ser. No. 14/615,038, entitled “Analysis of
135. Cardiac Rhythm Using RR Interval Characterization”, filed Feb. 5, 2015.
136 U.S. Pat. No. 9,962,101, issued May 8, 2018.
137. U.S. Utility patent application Ser. No. 12/594,842, entitled “Method,
138. System and Computer Program Product for Non-Invasive Classification of Cardiac Rhythm”, filed Oct. 6, 2009.
139. U.S. Pat. No. 8,983,584, issued Mar. 17, 2015.
140. International Patent Application Serial No. PCT/US2008/060021, entitled “Method, System and Computer Program Product for Non-Invasive Classification of Cardiac Rhythm”, filed Apr. 11, 2008; Publication No. WO2008/128034, Oct. 23, 2008.
141. U.S. Utility patent application Ser. No. 15/877,051, entitled “SYSTEM, METHOD AND COMPUTER PROGRAM PRODUCT FOR THE ORGANISM-SPECIFIC DIAGNOSIS OF SEPTICEMIA IN INFANTS”, filed Jan. 22, 2018. Publication No. US-2018-0327805-A1, Nov. 15, 2018.
142. U.S. Utility patent application Ser. No. 13/695,243, entitled “SYSTEM, METHOD AND COMPUTER PROGRAM PRODUCT FOR THE ORGANISM-SPECIFIC DIAGNOSIS OF SEPTICEMIA IN INFANTS”, filed Oct. 29, 2012; Publication No. 2013/0203044, Aug. 8, 2013.
143. International Patent Application Serial No. PCT/US2011/034487, entitled “SYSTEM, METHOD AND COMPUTER PROGRAM PRODUCT FOR THE ORGANISMSPECIFIC DIAGNOSIS OF SEPTICEMIA IN INFANTS”, filed Apr. 29, 2011; Publication No. WO 2011/137306, Nov. 3, 2011.
144. U.S. Utility patent application Ser. No. 15/319,270, entitled “CONTINUOUS MONITORING OF EVENT TRAJECTORIES SYSTEM AND RELATED METHOD”, filed Dec. 15, 2016; Publication No. US-2017-0147776-A1, May 25, 2017.
145. International Patent Application Serial No. PCT/US2015/036215, entitled “CONTINUOUS MONITORING OF EVENT TRAJECTORIES SYSTEM AND RELATED METHOD”, filed Jun. 17, 2015; Publication No. WO2015195793, Dec. 23, 2015.
146. U.S. Utility patent application Ser. No. 14/900,448, entitled “Multidimensional Time Series Entrainment System, Method and Computer Readable Medium”, filed Dec. 21, 2015; Publication No. US-2016-0143594-A1, May 26, 2016.
147. International Patent Application Serial No. PCT/US2014/043043, entitled “Multidimensional Time Series Entrainment System, Method and Computer Readable Medium”, filed Jun. 18, 2014; Publication No. WO2014205139, Dec. 24, 2014.
148. U.S. Utility patent application Ser. No. 14/400,408, entitled “Ventricular Activation (RR) Entropy Change As a Predictor of Sudden Cardiac Death in Cardiac Resynchronization Therapy Patients”, filed Nov. 11, 2014.
149. U.S. Pat. No. 9,839,364, issued Dec. 12, 2017.
150. International Patent Application Serial No. PCT/US2013/040677, entitled “Ventricular Activation (RR) Entropy Change As a Predictor of Sudden Cardiac Death in Cardiac Resynchronization Therapy Patients”, filed May 11, 2013; Publication No. WO 2013/170232, Nov. 14, 2013.
151. U.S. Utility patent application Ser. No. 14/064,853, entitled “System, Method and Computer Program Product for Detection of Changes in Health Status and Risk of Imminent Illness”, filed Oct. 28, 2013; Publication No. 2014/0052011, Feb. 20, 2014.
152. U.S. Utility patent application Ser. No. 12/866,056, entitled “System, Method and Computer Program Product for Detection of Changes in Health Status and Risk of Imminent Illness”, filed Aug. 4, 2010.
153. U.S. Pat. No. 8,588,908, issued Nov. 19, 2013.
154. International Patent Application Serial No. PCT/US2009/033082, entitled “System, Method and Computer Program Product for Detection of Changes in Health Status and Risk of Imminent Illness”, filed Feb. 4, 2009, Publication No. WO 2009/100133, Aug. 13, 2009.
155. U.S. Utility patent application Ser. No. 12/724,162, entitled “METHOD, SYSTEM AND COMPUTER PROGRAM METHOD FOR DETECTION OF PATHOLOGICAL FLUCTUATIONS OF PHYSIOLOGICAL SIGNALS TO DIAGNOSE HUMAN ILLNESS”, filed Mar. 15, 2010.
156. U.S. Pat. No. 8,615,291, issued Dec. 24, 2013.
157. U.S. Utility patent application Ser. No. 10/545,257, entitled “Quantitative Fetal Heart Rate and Cardiotocographic Monitoring System and Related Method Thereof”, ffiled Aug. 10, 2005.
158. U.S. Pat. No. 7,519,417, issued Apr. 14, 2009.
159. International Patent Application Serial No. US2004/004113, entitled “Quantitative Fetal Heart Rate and Cardiotocographic Monitoring System and Related Method Thereof”, filed Feb. 12, 2004; Publication No. WO04072822, Aug. 26, 2004.
160. U.S. Utility patent application Ser. No. 11/000,630, entitled “METHOD AND APPARATUS FOR THE EARLY DIAGNOSIS OF SUBACUTE, POTENTIALLY CATASTROPHIC ILLNESS”, filed Dec. 1, 2004.
161. U.S. Pat. No. 7,774,050, issued Aug. 10, 2010.
162. U.S. Utility patent application Ser. No. 09/770,653, entitled “METHOD FOR THE EARLY DIAGNOSIS OF SUBACUTE, POTENTIALLY CATASTROPHIC ILLNESS”, filed Jan. 29, 2001; Publication No. 2003/0100841, May 29, 2003.
163. U.S. Utility patent application Ser. No. 09/668,309, entitled “METHOD AND APPARATUS FOR THE EARLY DIAGNOSIS OF SUBACUTE, POTENTIALLY CATASTROPHIC ILLNESS”, filed Sep. 25, 2000.
164. International Patent Application Serial No. US99/05687, entitled “METHOD AND APPARATUS FOR THE EARLY DIAGNOSIS OF SUBACUTE, POTENTIALL CATASTROPHIC ILLNESS”, filed Mar. 17, 1999; Publication No. WO99/47040, Sep. 23, 1999.
165. U.S. Utility patent application Ser. No. 09/271,279, entitled “METHOD AND APPARATUS FOR THE EARLY DIAGNOSIS OF SUBACUTE, POTENTIALLY CATASTROPHIC ILLNESS”, filed Mar. 17, 1999
166. International Patent Application Serial No. PCT/US2002/005676, entitled “METHOD AND APPARATUS FOR THE EARLY DIAGNOSIS OF SUBACUTE, POTENTIALLY CATASTROPHIC ILLNESS”, filed Feb. 27, 2002; Publication No. WO02/67776, Sep. 6, 2002.
167. U.S. Utility patent application Ser. No. 09/793,653, entitled “METHOD AND APPARATUS FOR THE EARLY DIAGNOSIS OF SUBACUTE, POTENTIALLY CATASTROPHIC ILLNESS”, filed Feb. 27, 2001; Publication No. 2002/0052557, May 2, 2002.
168. U.S. Utility patent application Ser. No. 10/069,674, entitled “Method and Apparatus for Predicting the Risk of Hypoglycemia”, filed Feb. 22, 2002.
169. U.S. Pat. No. 6,923,763, issued Aug. 2, 2005.
170. International Patent Application Serial No. US00/22886, entitled “METHOD AND APPARATUS FOR PREDICTING THE RISK OF HYPOGLYCEMIA”, filed Aug. 21, 2000; Publication No. WO01/13786, Mar. 1, 2001.
171. U.S. Utility patent application Ser. No. 15/319,240, entitled “SYSTEM AND METHOD FOR IMPROVED CARDIAC RHYTHM CLASSIFICATION FROM TIME BETWEEN HEART BEAT INTERVALS USING NON-LINEAR DYNAMICS”, filed Dec. 15, 2016; Publication No. US-2017-0127964-A1, May 11, 2017.
172. International Patent Application Serial No. PCT/US2015/036238, entitled “SYSTEM AND METHOD FOR IMPROVED CARDIAC RHYTHM CLASSIFICATION FROM TIME BETWEEN HEART BEAT INTERVALS USING NON-LINEAR DYNAMICS”, filed Jun. 17, 2015; Publication No. WO2015195806, Dec. 23, 2015.

What is claimed is:

1. A system for predicting hypoglycemia risk, the system comprising:
a processor including instructions to cause the processor to:
receive a first set of patient data;
apply data processing to identify features of the first set of patient data that are associated with hypoglycemia;
apply multivariable modeling to the features to generate a multivariable model that outputs a risk score associated with future hypoglycemia where the multivariable model captures a pathophysiological signature of impending hypoglycemia;
receive a second set of patient data, apply data processing to identify features of the second set of patient data, and apply the multivariable model to generate a risk score for the second set of patient data;
analyze the risk score of the second set of patient data to determine an appropriate clinical decision support; and
output a result for access by a device;
wherein at least one or more of the first set of patient data and/or the second set of patient data are representative of a physiological measurement from at least one or more of a cardiorespiratory monitoring (CRM) data source, an electronic medical record (EMR) vital sign data source, and/or a biochemical laboratory (LAB) data source.

2. The system of claim 1, wherein:
at least one or more of the first set of patient data and/or the second set of patient data includes a biochemical measurement.

3. The system of claim 1, wherein:
the physiological measurement includes waveform data related to heart rate and/or respiratory rate.

4. The system of claim 1, wherein:
the multivariable modeling includes at least one or more of: logistic regression, random forest, xgboost, support vector machines, nearest neighbor, artificial neural networks, and/or long short-term memory (LSTM).

5. The system of claim 1, comprising:
the processor is configure to analyze at least one or more of the features of the first set of patient data and/or the features of the second set of patient data to identify feature importance measures representative of the a feature's association with hypoglycemia.

6. The system of claim 1, comprising the processor in combination with at least one or more of a glycemic state monitoring device, a glucose management system, and/or an insulin recommendation system, wherein:
the processor is configured to generate a signal to inform the glycemic state monitoring device, the glucose management system, and/or the insulin recommendation system about hypoglycemia risk based on the analysis of the risk score.

7. The system of claim 6, wherein:
the processor is configured to generate the signal that includes a notification communication recommending, based on the analysis of the risk score, at least one or more of: risk of hypoglycemia, change in risk of hypoglycemia, check patient glucose level, modification of insulin dosage, modification of basal insulin, modification of basal insulin rate, modification of insulin infusion rate, and/or modification of patient nutritional administration.

8. The system of claim 7, wherein:
the processor is configured to generate the notification communication recommending modification of insulin infusion rate as a glucose clamp, wherein blood glucose is maintained within a range so as to bound blood glucose to an upper level and/or a lower level.

9. The system of claim 1, comprising the processor in combination with an insulin delivery device, wherein:
t
the processor is configured to generate a signal to inform the insulin delivery device about hypoglycemia risk based on the analysis of the risk score.

10. The system of claim 9, wherein:
the processor is configured to generate the signal that includes a command signal requiring, based on the analysis of risk, at least one or more of: risk of hypoglycemia, change in risk of hypoglycemia, check patient glucose level, modification of insulin dosage, modification of basal insulin, modification of basal insulin rate, modification of insulin infusion rate, and/or modification of patient nutritional administration.

11. The system of claim 10, wherein:
the processor is configured to generate the command signal requiring modification of insulin infusion rate as a glucose clamp, wherein blood glucose is maintained within a range so as to bound blood glucose to an upper level and/or a lower level.

12. The system of claim 1, comprising the processor in combination with a data store,
wherein:
the data store is configured to contain plural multivariable models.

13. The system of claim 12, wherein:
the processor is configured to select the multivariable model for implementation from the plural multivariable models based on at least one or more of: a type of first set patient data and/or a type of second set patient data.

14. The system of claim 12, wherein:
the processor is configured to switch from a first multivariable model to a second multivariable model for implementation based on at least one or more of: a type of first set patient data and/or a type of second set patient data.

15. The system of claim 12, wherein:
the plural multivariable models include at least one or more of:
a CRM data model;
an EMR vital sign data model;
a LAB data model;
a CRM/EMR vital sign data model;
a CRM/LAB data model;
an EMR vital sign/LAB data model; and/or
a CRM/EMR vial sign/LAB data model.

16. The system of claim 1, wherein:
the processor is configured to update the multivariable model based at least one or more of: a type of first set patient data and/or a type of second set patient data.

17. A method for predicting hypoglycemia risk, the method comprising:
receiving a first set of patient data;
applying data processing to identify features of the first set of patient data that are associated with hypoglycemia;
applying multivariable modeling to the features to generate a multivariable model that outputs a risk score associated with future hypoglycemia where the multivariable model captures a pathophysiological signature of impending hypoglycemia;
receiving a second set of patient data, applying data processing to identify features of the second set of patient data, and applying the multivariable model to generate a risk score for the second set of patient data;
analyzing the risk score of the second set of patient data to determine an appropriate clinical decision support; and
outputting a result for access by a device;
wherein at least one or more of the first set of patient data and/or the second set of patient data are representative of a physiological measurement from at least one or more of a cardiorespiratory monitoring (CRM) data source, an electronic medical record (EMR) vital sign data source, and/or a biochemical laboratory (LAB) data source.

18. The method of claim 17, wherein:
the multivariable modeling includes at least one or more of: logistic regression, random forest, xgboost, support vector machines, nearest neighbor, artificial neural networks, and/or long short-term memory (LSTM).

19. A computer readable medium having instructions stored thereon that when executed by a processor causes the processor to predict hypoglycemia risk by:
receiving a first set of patient data;
applying data processing to identify features of the first set of patient data that are associated with hypoglycemia;
applying multivariable modeling to the features to generate a multivariable model that outputs a risk score associated with future hypoglycemia where the multivariable model captures a pathophysiological signature of impending hypoglycemia;
receiving a second set of patient data, applying data processing to identify features of the second set of patient data, and applying the multivariable model to generate a risk score for the second set of patient data;
analyzing the risk score of the second set of patient data to determine an appropriate clinical decision support; and
outputting a result for access by a device;
wherein at least one or more of the first set of patient data and/or the second set of patient data are representative of a physiological measurement from at least one or more of a cardiorespiratory monitoring (CRM) data source, an electronic medical record (EMR) vital sign data source, and/or a biochemical laboratory (LAB) data source.

20. The computer readable medium of claim 20, wherein:
the multivariable modeling includes at least one or more of: logistic regression, random forest, xgboost, support vector machines, nearest neighbor, artificial neural networks, and/or long short-term memory (LSTM).
